(19) United States
(12) Patent Application Publication
(10) Pub. No.: US 2007/0116710 A1
(43) Pub. Date: May 24, 2007

(54) Methods of treating hemolytic anemia

(76) Inventors: Leonard Bell, Woodbridge, CT (US); Russell P. Rother, Prospect, CT (US)

Correspondence Address: FISH & NEAVE IP GROUP;ROPES & GRAY LLP, ONE INTERNATIONAL PLACE, BOSTON, MA, 02110-2624, US

(21) Appl. No.: 11/595,118

(22) Filed: Nov. 8, 2006

Related U.S. Application Data
(63) Continuation-in-part of application No. 11/050,543, filed on Feb. 3, 2005.
Application No. 11/050,543 is a continuation-in-part of application No. 10/771,552, filed on Feb. 3, 2004.
(60) Provisional application No. 60/783,070, filed on Mar. 15, 2006.

(51) Int. Cl. A61K 39/00; A61K 48/00

(52) U.S. Cl. 424/185.1; 514/44

(57) ABSTRACT

Paroxysmal nocturnal hemoglobinuria or other hemolytic diseases are treated using a compound which binds to or otherwise blocks the generation and/or the activity of one or more complement components, such as, for example, a complement-inhibiting antibody.

### RELATED APPLICATIONS

This application is a continuation-in-part of U.S. patent application Ser. No. 11/050,543, filed Feb. 3, 2005, which is a continuation-in-part of U.S. patent application Ser. No. 10/771,552, filed Feb. 3, 2004, and further claims the benefit of U.S. provisional patent application Ser. No. 60/783,070, filed Mar. 15, 2006, the entire disclosures of which are incorporated herein by this reference.

### BACKGROUND

1. Technical Field

This disclosure relates to a method of treating a hemolytic disease such as, for example, paroxysmal nocturnal hemoglobinuria (“PNH”), by administering a compound which binds to, or otherwise blocks, the generation and/or activity of one or more complement components.

2. Background of Related Art

Paroxysmal nocturnal hemoglobinuria (“PNH”) is an uncommon blood disorder wherein red blood cells are compromised and are thus destroyed more rapidly than normal red blood cells. PNH results from a mutation of bone marrow cells resulting in the generation of abnormal blood cells. More specifically, PNH is believed to be a disorder of hematopoietic stem cells, which give rise to distinct populations of mature blood cells. The basis of the disease appears to be somatic mutations leading to the inability to synthesize the glycosyl-phosphatidylinositol (“GPI”) anchor that is responsible for attaching proteins to cell membranes. The mutated gene, PIG-A (phosphatidylinositol glycan class A) resides in the X chromosome and can have several different mutations, varying from deletions to point mutations.

PNH causes a sensitivity to complement-mediated destruction and this sensitivity occurs in the cell membrane. PNH cells are deficient in a number of proteins, particularly essential complement-regulating surface proteins. These complement-regulating surface proteins include the decay-accelerating factor (“DAF”) or CD55 and membrane inhibitor of reactive lysis (“MIRL”) or CD59.

PNH is characterized by hemolytic anemia (a decreased number of red blood cells) and hemoglobinuria (excess hemoglobin in the urine). PNH-afflicted individuals are known to have paroxysms, which are defined here as an exacerbation of hemolysis with dark-colored urine. Hemolytic anemia is due to intravascular destruction of red blood cells by complement components. Other known symptoms include dysphagia, fatigue, erectile dysfunction, thrombosis, recurrent abdominal pain, pulmonary hypertension, and an overall poor quality of life.

Hemolysis resulting from intravascular destruction of red blood cells causes local and systemic nitric oxide (NO) deficiency through the release of free hemoglobin. Free hemoglobin is a very efficient scavenger of NO, due in part to the accessibility of NO in the non-erythrocyte compartment and a 10⁶ times greater affinity of the heme moiety for NO than that for oxygen. The occurrence of intravascular hemolysis often generates sufficient free hemoglobin to completely deplete haptoglobin. Once the capacity of this hemoglobin scavenging protein is exceeded, consumption of endogenous NO ensues. For example, in a setting of intravascular hemolysis such as PNH, where LDH levels routinely exceed the upper limit of the normal range and commonly reach levels of 2-3 times their normal levels, free hemoglobin would likely obtain concentrations of 0.8-1.6 g/L. Since haptoglobin can only bind somewhere between 0.7 to 1.5 g/L of hemoglobin depending on the haptoglobin allotype, a large excess of free hemoglobin would be generated. Once the capacity of hemoglobin reabsorption by the kidney proximal tubules is exceeded, hemoglobinuria ensues. The release of free hemoglobin during intravascular hemolysis results in excessive consumption of NO with subsequent enhanced smooth muscle contraction, vasoconstriction and platelet activation and aggregation. PNH-related morbidities associated with NO scavenging by hemoglobin include abdominal pain, erectile dysfunction, esophageal spasm, and thrombosis.

The laboratory evaluation of hemolysis normally includes hematologic, serologic, and urine tests. Hematologic tests include an examination of the blood smear for morphologic abnormalities of red blood cells (RBCs) (to determine causation), and the measurement of the reticulocyte count in whole blood (to determine bone marrow compensation for RBC loss). Serologic tests include lactate dehydrogenase (LDH; widely performed), and free hemoglobin (not widely performed) as a direct measure of hemolysis. LDH levels can be useful in the diagnosis and monitoring of patients with hemolysis. Other serologic tests include bilirubin or haptoglobin, as measures of breakdown products or scavenging reserve, respectively. Urine tests include bilirubin, hemosiderin, and free hemoglobin, and are generally used to measure gross severity of hemolysis and for differentiation of intravascular vs. extravascular etiologies of hemolysis rather than routine monitoring of hemolysis. Further, RBC numbers, RBC (i.e. cell-bound) hemoglobin, and hematocrit are generally performed to determine the extent of any accompanying anemia rather than as a measure of hemolytic activity per se.

Steroids have been employed as a therapy for hemolytic diseases and may be effective in suppressing hemolysis in some patients, although long term use of steroid therapy carries many negative side effects. Afflicted patients may require blood transfusions, which carry risks of infection. Anti-coagulation therapy may also be required to prevent blood clot formation and can result in hemorrhage. Bone marrow transplantation has been known to cure PNH, however, bone marrow matches are often very difficult to find and mortality rates are high with this procedure.

It would be advantageous to provide a treatment which safely and reliably eliminates and/or limits hemolytic diseases, such as PNH, and their effects.

### SUMMARY

In one aspect, the application provides a method of reducing the occurrence of thrombosis in a subject, said method comprising inhibiting complement in said subject. In certain embodiments, the method comprises administering a compound to said subject, wherein the compound is selected from the group consisting of: a) compounds which bind to one or more complement components, b) compounds which block the generation of one or more complement components, and c) compounds which block the activity of one or more complement components.

In certain embodiments, the subject has a paroxysmal nocturnal hemoglobinuria (PNH) granulocyte clone greater than 0.1% of the total granulocyte count. In certain embodiments, the subject has a PNH granulocyte clone greater than 1% of the total granulocyte count. In certain embodiments, the subject has a PNH granulocyte clone greater than 10% of the total granulocyte count. In certain embodiments, the subject has a PNH granulocyte clone greater than 50% of the total granulocyte count.

In certain embodiments, the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

In certain embodiments, the compound is selected from the group consisting of CR1, LEX-CRI, MCP, DAF, CD59, Factor H, cobra venom factor, FUT-175, complestatin, and K76 COOH.

In certain embodiments, the compound inhibits C5b activity. In certain embodiments, the compound inhibits cleavage of C5. In certain embodiments, the compound inhibits terminal complement. In certain embodiments, the compound inhibits C5a activity or inhibits binding of C5a to its receptor.

In certain embodiments, the subject is a human. In certain embodiments, the subject has a history of one or more thrombotic events.

In certain embodiments, the compound is an antibody or antibody fragment. In certain embodiments, the antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

In certain embodiments, the antibody is pexelizumab. In certain embodiments, the antibody is eculizumab.

In certain embodiments, the compound is administered chronically to said subject. In certain embodiments, the compound is administered systemically to said subject. In certain embodiments, the compound is administered locally to said subject.

In certain embodiments, the method reduces rates of thromboembolism by greater than 25%. In certain embodiments, the method reduces rates of thromboembolism by greater than 50%. In certain embodiments, the method reduces rates of thromboembolism by greater than 75%. In certain embodiments, the method reduces rates of thromboembolism by greater than 90%.

In certain embodiments, the method results in at least a 25% reduction in LDH levels. In certain embodiments, the method results in at least a 50% reduction in LDH levels. In certain embodiments, the method results in at least a 75% reduction in LDH levels. In certain embodiments, the method in a subject results in at least a 90% reduction in LDH levels.

In certain embodiments, the method further comprising administering a second compound, wherein said second compound increases hematopoiesis. In certain embodiments, the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG). In certain embodiments, EPO is administered with an anti-C5 antibody. In certain embodiments, the antibody is pexelizumab. In certain embodiments, the antibody is eculizumab.

In certain embodiments, the method further comprising administering an antithrombotic compound. In certain embodiments, the antithrombotic compound is an anticoagulant. In certain embodiments, the anticoagulant is administered with an anti-C5 antibody. In certain embodiments, the anticoagulant is an antiplatelet agent. In certain embodiments, the antibody is pexelizumab. In certain embodiments, the antibody is eculizumab.

In another aspect, the application provides a method of reducing the occurrence of thrombosis in a subject who has a higher than normal lactate dehydrogenase (LDH) level, said method comprising inhibiting complement in said subject.

In certain embodiments, the method comprises administering a compound to said subject, wherein the compound is selected from the group consisting of: a) compounds which bind to one or more complement components, b) compounds which block the generation of one or more complement components, and c) compounds which block the activity of one or more complement components.

In certain embodiments, the subject has an LDH level greater than the upper limit of normal. In certain embodiments, the subject has an LDH level greater than or equal to 1.5 times the upper limit of normal. In certain embodiments, the subject has an LDH level greater than or equal to 2.5 times the upper limit of normal. In certain embodiments, the subject has an LDH level greater than or equal to 5 times the upper limit of normal. In certain embodiments, the subject has an LDH level greater than or equal to 10 times the upper limit of normal.

In certain embodiments, the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

In certain embodiments, the compound is selected from the group consisting of CR1, LEX-CR1, MCP, DAF, CD59, Factor H, cobra venom factor, FUT-175, complestatin, and K76 COOH.

In certain embodiments, the compound inhibits C5b activity. In certain embodiments, the compound inhibits cleavage of C5. In certain embodiments, the compound inhibits terminal complement. In certain embodiments, the compound inhibits C5a activity or inhibits binding of C5a to its receptor.

In certain embodiments, the subject is a human. In certain embodiments, the subject has a history of one or more thrombotic events.

In certain embodiments, the compound is an antibody or antibody fragment. In certain embodiments, the antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

In certain embodiments, the antibody is pexelizumab. In certain embodiments, the antibody is eculizumab.

In certain embodiments, the compound is administered chronically to said subject. In certain embodiments, the compound is administered systemically to said subject. In certain embodiments, the compound is administered locally to said subject.

In certain embodiments, the method reduces rates of thromboembolism by greater than 25%. In certain embodiments, the method reduces rates of thromboembolism by greater than 50%. In certain embodiments, the method reduces rates of thromboembolism by greater than 75%. In certain embodiments, the method reduces rates of thromboembolism by greater than 90%.

In certain embodiments, the method results in at least a 25% reduction in LDH levels. In certain embodiments, the method results in at least a 50% reduction in LDH levels. In certain embodiments, the method results in at least a 75% reduction in LDH levels. In certain embodiments, the method in a subject results in at least a 90% reduction in LDH levels.

In certain embodiments, the method further comprising administering a second compound, wherein said second compound increases hematopolesis. In certain embodiments, the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG). In certain embodiments, EPO is administered with an anti-C5 antibody. In certain embodiments, the antibody is pexelizumab. In certain embodiments, the antibody is eculizumab.

In certain embodiments, the method further comprising administering an antithrombotic compound. In certain embodiments, the antithrombotic compound is an anticoagulant. In certain embodiments, the anticoagulant is administered with an anti-C5 antibody. In certain embodiments, the anticoagulant is an antiplatelet agent. In certain embodiments, the antibody is pexelizumab. In certain embodiments, the antibody is eculizumab.

In still another aspect, the application provides a method of reducing the occurrence of thrombosis in a subject who has a PNH granulocyte clone and an LDH level greater than the upper limit of normal, said method comprising inhibiting complement in said subject. In certain embodiments, the method comprises administering a compound to said subject, wherein the compound is selected from the group consisting of: a) compounds which bind to one or more complement components, b) compounds which block the generation of one or more complement components, and c) compounds which block the activity of one or more complement components.

In certain embodiments, the subject has a PNH granulocyte clone greater than 0.1% of the total granulocyte count. In certain embodiments, the subject has a PNH granulocyte clone greater than 0.1% of the total granulocyte count. In certain embodiments, the subject has a PNH granulocyte clone greater than 1% of the total granulocyte count. In certain embodiments, the subject has a PNH granulocyte clone greater than 10% of the total granulocyte count. In certain embodiments, the subject has a PNH granulocyte clone greater than 50% of the total granulocyte count.

In certain embodiments, the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

In certain embodiments, the compound is selected from the group consisting of CR1, LEX-CR1, MCP, DAF, CD59, Factor H, cobra venom factor, FUT-175, complestatin, and K76 COOH.

In certain embodiments, the compound inhibits C5b activity. In certain embodiments, the compound inhibits cleavage of C5. In certain embodiments, the compound inhibits terminal complement. In certain embodiments, the compound inhibits C5a activity or inhibits binding of C5a to its receptor.

In certain embodiments, the subject is a human. In certain embodiments, the subject has a history of one or more thrombotic events.

In certain embodiments, the compound is an antibody or antibody fragment. In certain embodiments, the antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

In certain embodiments, the antibody is pexelizumab. In certain embodiments, the antibody is eculizumab.

In certain embodiments, the compound is administered chronically to said subject. In certain embodiments, the compound is administered systemically to said subject. In certain embodiments, the compound is administered locally to said subject.

In certain embodiments, the method reduces rates of thromboembolism by greater than 25%. In certain embodiments, the method reduces rates of thromboembolism by greater than 50%. In certain embodiments, the method reduces rates of thromboembolism by greater than 75%. In certain embodiments, the method reduces rates of thromboembolism by greater than 90%.

In certain embodiments, the method results in at least a 25% reduction in LDH levels. In certain embodiments, the method results in at least a 50% reduction in LDH levels. In certain embodiments, the method results in at least a 75% reduction in LDH levels. In certain embodiments, the method in a subject results in at least a 90% reduction in LDH levels.

In certain embodiments, the method further comprising administering a second compound, wherein said second compound increases hematopoiesis. In certain embodiments, the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG). In certain embodiments, EPO is administered with an anti-C5 antibody. In certain embodiments, the antibody is pexelizumab. In certain embodiments, the antibody is eculizumab.

In certain embodiments, the method further comprising administering an antithrombotic compound. In certain embodiments, the antithrombotic compound is an anticoagulant. In certain embodiments, the anticoagulant is administered with an anti-C5 antibody. In certain embodiments, the anticoagulant is an antiplatelet agent. In certain embodiments, the antibody is pexelizumab. In certain embodiments, the antibody is eculizumab.

In yet another aspect, the application provides a method of reducing the occurrence of thrombosis in a subject suffering from a lower than normal nitric oxide (NO) level, said method comprising inhibiting complement in said subject. In certain embodiments, the method comprises administering a compound to said subject, wherein the compound is selected from the group consisting of: i) compounds which bind to one or more complement components, ii) compounds which block the generation of one or more complement components, and iii) compounds which block the activity of one or more complement components, wherein said method increases serum nitric oxide (NO) levels.

In certain embodiments, the method increases NO levels by greater than 25%. In certain embodiments, the method increases NO levels by greater than 50%. In certain embodiments, the method increases NO levels by greater than 100%. In certain embodiments, the method increases NO levels by greater than 3 fold.

In certain embodiments, the subject has PNH.

In certain embodiments, the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

In certain embodiments, the compound is selected from the group consisting of CR1, LEX-CR1, MCP, DAF, CD59, Factor H, cobra venom factor, FUT-175, complestatin, and K76 COOH.

In certain embodiments, the compound inhibits C5b activity. In certain embodiments, the compound inhibits cleavage of C5. In certain embodiments, the compound inhibits terminal complement. In certain embodiments, the compound inhibits C5a activity or inhibits binding of C5a to its receptor.

In certain embodiments, the subject is a human. In certain embodiments, the subject has a history of one or more thrombotic events.

In certain embodiments, the compound is an antibody or antibody fragment. In certain embodiments, the antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

In certain embodiments, the antibody is pexelizumab. In certain embodiments, the antibody is eculizumab.

In certain embodiments, the compound is administered chronically to said subject. In certain embodiments, the compound is administered systemically to said subject. In certain embodiments, the compound is administered locally to said subject.

In certain embodiments, the method reduces rates of thromboembolism by greater than 25%. In certain embodiments, the method reduces rates of thromboembolism by greater than 50%. In certain embodiments, the method reduces rates of thromboembolism by greater than 75%. In certain embodiments, the method reduces rates of thromboembolism by greater than 90%.

In certain embodiments, the method results in at least a 25% reduction in LDH levels. In certain embodiments, the method results in at least a 50% reduction in LDH levels. In certain embodiments, the method results in at least a 75% reduction in LDH levels. In certain embodiments, the method in a subject results in at least a 90% reduction in LDH levels.

In certain embodiments, the method further comprising administering a second compound, wherein said second compound increases hematopoiesis. In certain embodiments, the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG). In certain embodiments, EPO is administered with an anti-C5 antibody. In certain embodiments, the antibody is pexelizumab. In certain embodiments, the antibody is eculizumab.

In certain embodiments, the method further comprising administering an antithrombotic compound. In certain embodiments, the antithrombotic compound is an anticoagulant. In certain embodiments, the anticoagulant is administered with an anti-C5 antibody. In certain embodiments, the anticoagulant is an antiplatelet agent. In certain embodiments, the antibody is pexelizumab. In certain embodiments, the antibody is eculizumab.

In another aspect, the application provides a method of determining whether a subject having a hemolytic disorder is susceptible to thrombosis comprising measuring the PNH granulocyte clone size of said subject, wherein if the clone size is greater than 0.1% then said subject is susceptible to thrombosis. In certain embodiments, the clone size is greater than 1%. In certain embodiments, the clone size is greater than 10%. In certain embodiments, the clone size is greater than 50%.

In still another aspect, the application provides a method of increasing PNH red blood cell mass of a subject, said method comprising inhibiting complement in said subject. In certain embodiments, the method comprises administering a compound to the subject, the compound being selected from the group consisting of: i) compounds which bind to one or more complement components, ii) compounds which block the generation of one or more complement components, and iii) compounds which block the activity of one or more complement components.

In certain embodiments, the subject has a PNH granulocyte clone. In certain embodiments, the PNH granulocyte clone is greater than 0.1% of the total granulocyte count. In certain embodiments, the PNH granulocyte clone is greater than 1% of the total granulocyte count. In certain embodiments, the PNH granulocyte clone is greater than 10% of the total granulocyte count. In certain embodiments, the PNH granulocyte clone is greater than 50% of the total granulocyte count.

In certain embodiments, the subject has an LDH level greater than the upper limit of normal. In certain embodiments, the subject has an LDH level greater than or equal to 1.5 times the upper limit of normal. In certain embodiments, the subject has an LDH level greater than or equal to 2.5 times the upper limit of normal. In certain embodiments, the subject has an LDH level greater than or equal to 5 times the upper limit of normal. In certain embodiments, the subject has an LDH level greater than or equal to 10 times the upper limit of normal.

In yet another aspect, the application provides a method of treating hemolytic anemia in a subject, said method comprising inhibiting complement in said subject. In certain embodiments, the method comprises administering a compound to the subject, wherein the compound is selected from the group consisting of: i) compounds which bind to one or more complement components, ii) compounds which block the generation of one or more complement components, and iii) compounds which block the activity of one or more complement components, wherein said method increases red blood cell (RBC) mass.

In certain embodiments, the RBC mass is measured as the absolute number of RBCs. In certain embodiments, the RBC mass is PNH RBC mass. In certain embodiments, the method increases RBC mass by greater than 10%. In certain embodiments, the method increases RBC mass by greater than 25%. In certain embodiments, the method increases RBC mass by greater than 50%. In certain embodiments, the method increases RBC mass by greater than 100%. In certain embodiments, the method increases RBC mass by greater than 2 fold.

In certain embodiments, the method decreases transfusion requirements.

In certain embodiments, the method stabilizes hemoglobin levels.

In certain embodiments, the method causes an increase in hemoglobin levels.

The application contemplates combinations of any of the foregoing aspects and embodiments.

### BRIEF DESCRIPTION OF THE DRAWINGS

FIG. 1A reports biochemical parameters of hemolysis measured during treatment of PNH patients with an anti-C5 antibody.

FIG. 1B graphically depicts the effect of treatment with an anti-C5 antibody on lactate dehydrogenase (LDH) levels.

FIG. 2 shows a urine color scale devised to monitor the incidence of paroxysm of hemoglobinuria in PNH patients.

FIG. 3 is a graph of the effects of eculizumab treatments on patient paroxysm rates, as compared to pre-treatment rates.

FIG. 4 shows urine samples of PNH patients and measurements of hemoglobinuria, dysphagia, LDH, AST, pharmacokinetics (PK) and pharmacodynamics (PD) reflecting the immediate and positive effects of the present methods on hemolysis, symptoms and pharmacodynamics suitable to completely block complement.

FIG. 5 graphically depicts the effect of anti-C5 antibody dosing schedule on hemoglobinuria over time.

FIGS. 6a and 6b are graphs comparing the number of transfusion units required per patient per month, prior to and during treatment with an anti-C5 antibody: FIG. 6a depicts cytopenic patients; and FIG. 6b depicts non-cytopenic patients.

FIG. 7 shows the management of a thrombocytopenic patient by administering an anti-C5 antibody and erythropoietin (EPO).

FIG. 8 graphically depicts the pharmacodynamics of an anti-C5 antibody.

FIG. 9 is a chart of the results of European Organization for Research and Treatment of Cancer questionnaires (“EORTC QLC-C30”) completed during the anti-C5 therapy regimen addressing quality of life issues.

FIG. 10 is a chart depicting the effects of anti-C5 antibody treatments on adverse symptoms associated with PNH.

FIG. 11 shows changes in PNH RBC mass during treatment with eculizumab compared with placebo.

FIG. 12 shows the effect of eculizumab and recombinant human erythropoietin on PNH Type III RBC mass and transfusion requirements. The diamonds represent PNH type III RBC counts and solid bars represent the number of packed red blood cell (PRBC) units transfused. The x-axis indicates date.

FIG. 13 shows changes in FACIT-Fatigue score during treatment with eculizumab and for placebo control.

### DETAILED DESCRIPTION

The present disclosure relates to a method of treating paroxysmal nocturnal hemoglobinuria (“PNH”) and other hemolytic diseases in marnmals. Specifically, the methods of treating hemolytic diseases, which are described herein, involve using compounds which bind to or otherwise block the generation and/or activity of one or more complement components. The present methods have been found to provide surprising results. For instance, hemolysis rapidly ceases upon administration of the compound which binds to or otherwise blocks the generation and/or activity of one or more complement components, with LDH and hemoglobinuria being significantly reduced immediately after treatment. Also, hemolytic patients can be rendered less dependent on transfusions or transfusion-independent for extended periods (twelve months or more), well beyond the 120 day life cycle of red blood cells. In addition, type III red blood cell count can be increased dramatically in the midst of other mechanisms of red blood cell lysis (non-complement mediated and/or earlier complement component mediated e.g., Cb3). Another example of a surprising result is that a variety of symptoms resolved, indicating that NO serum levels were increased enough even in the presence of other mechanisms of red blood cell lysis. These and other results reported herein are unexpected and could not be predicted from prior treatments of hemolytic diseases.

Any compound which binds to or otherwise blocks the generation and/or activity of one or more complement components can be used in the present methods. A specific class of such compounds which is particularly useful includes antibodies specific to a human complement component, especially anti-C5 antibodies. The anti-C5 antibody inhibits the complement cascade and, ultimately, prevents red blood cell (“RBC”) lysis by the terminal complement complex C5b-9. By inhibiting and/or reducing the lysis of RBCs, the effects of PNH and other hemolytic diseases (including symptoms such as hemoglobinuria, anemia, dysphagia, fatigue, erectile dysfunction, recurrent abdominal pain and thrombosis) are eliminated or decreased.

In another embodiment, soluble forms of the proteins CD55 and CD59, singularly or in combination with each other, can be administered to a subject to inhibit the complement cascade in its alternative pathway. CD55 inhibits at the level of C3, thereby preventing the further progression of the cascade. CD59 inhibits the C5b-8 complex from combining with C9 to form the membrane attack complex (see discussion below).

The complement system acts in conjunction with other immunological systems of the body to defend against intrusion of bacterial and viral pathogens. There are at least 25 proteins involved in the complement cascade, which are found as a complex collection of plasma proteins and membrane cofactors. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16th Edition.

The complement cascade progresses via the classical pathway, the alternative pathway or the lectin pathway. These pathways share many components, and while they differ in their initial steps, they converge and share the same “terminal complement” components (C5 through C9) responsible for the activation and destruction of target cells. The classical complement pathway is typically initiated by antibody recognition of and binding to an antigenic site on a target cell. The alternative pathway is usually antibody independent, and can be initiated by certain molecules on pathogen surfaces. Additionally, the lectin pathway is typically initiated with binding of mannose-binding lectin (“MBL”) to high mannose substrates. These pathways converge at the point where complement component C3 is cleaved by an active protease to yield C3a and C3b. C3a is an anaphylatoxin (see discussion below). C3b binds to bacteria and other cells, as well as to certain viruses and immune complexes, and tags them for removal from the circulation. C3b in this role is known as opsonin. The opsonic function of C3b is generally considered to be the most important anti-infective action of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone (Fearon, in Intensive Review of Internal Medicine, 2nd Ed. Fanta and Minaker, eds. Brigham and Women's and Beth Israel Hospitals, 1983).

C3b also forms a complex with other components unique to each pathway to form classical or alternative C5 convertase, which cleaves C5 into C5a and C5b. C3 is thus regarded as the central protein in the complement reaction sequence since it is essential to all three activation pathways (Wurzner, et al., Complement Inflamm. 1991, 8:328-340). This property of C3b is regulated by the serum protease Factor I, which acts on C3b to produce iC3b (inactive C3b). While still functional as an opsonin, iC3b can not form an active C5 convertase.

The pro-C5 precursor is cleaved after amino acid 655 and 659, to yield the beta chain as an amino terminal fragment (amino acid residues +1 to 655 of the sequence) and the alpha chain as a carboxyl terminal fragment (amino acid residues 660 to 1658 of the sequence), with four amino acids (amino acid residues 656-659 of the sequence) deleted between the two. C5 is glycosylated, with about 1.5-3 percent of its mass attributed to carbohydrate. Mature C5 is a heterodimer of a 999 amino acid 115 kDa alpha chain that is disulfide linked to a 655 amino acid 75 kDa beta chain. C5 is found in normal serum at approximately 75 μg/mL (0.4 μM). C5 is synthesized as a single chain precursor protein product of a single copy gene (Haviland et al., J. Immunol. 1991, 146:362-368). The cDNA sequence of the transcript of this gene predicts a secreted pro-C5 precursor of 1658 amino acids along with an 18 amino acid leader sequence (see, U.S. Pat. No. 6,355,245).

Cleavage of C5 releases C5a, a potent anaphylatoxin and chemotactic factor, and leads to the formation of the lytic terminal complement complex, C5b-9. C5a is cleaved from the alpha chain of C5 by either alternative or classical C5 convertase as an amino terminal fragment comprising the first 74 amino acids of the alpha chain (i.e., amino acid residues 660-733 of the sequence). Approximately 20 percent of the 11 kDa mass of C5a is attributed to carbohydrate. The cleavage site for convertase action is at, or immediately adjacent to, amino acid residue 733 of the sequence. A compound that binds at, or adjacent, to this cleavage site would have the potential to block access of the C5 convertase enzymes to the cleavage site and thereby act as a complement inhibitor.

C5b combines with C6, C7, and C8 to form the C5b-8 complex at the surface of the target cell. Upon binding of several C9 molecules, the membrane attack complex (“MAC”, C5b-9, terminal complement complex—TCC) is formed. When sufficient numbers of MACs insert into target cell membranes, the openings they create (MAC pores) mediate rapid osmotic lysis of the target cells. Lower, non-lytic concentrations of MACs can produce other proinflammatory effects. In particular, membrane insertion of small numbers of the C5b-9 complexes into endothelial cells and platelets can cause deleterious cell activation. In some cases activation may precede cell lysis.

C5a and C5b-9 also have pleiotropic cell activating properties, by amplifying the release of downstream inflammatory factors, such as hydrolytic enzymes, reactive oxygen species, arachidonic acid metabolites and various cytokines. C5 can also be activated by means other than C5 convertase activity. Limited trypsin digestion (Minta and Man, J. Immunol. 1977, 119:1597-1602; Wetsel and Kolb, J. Immunol. 1982, 128:2209-2216) and acid treatment (Yamamoto and Gewurz, J. Immunol. 1978, 120:2008; Damerau et al., Molec. Immunol. 1989, 26:1133-1142) can also cleave C5 and produce active C5b.

As mentioned above, C3a and C5a are anaphylatoxins. These activated complement components can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

Any compounds which bind to or otherwise block the generation and/or activity of any of the human complement components may be utilized in accordance with the present disclosure. In some embodiments, antibodies specific to a human complement component are useful herein. Some compounds include antibodies directed against complement components C-1, C-2, C-3, C-4, C-5, C-6, C-7, C-8, C-9, Factor D, Factor B, Factor P, MBL, MASP-1, and MASP-2, thus preventing the generation of the anaphylatoxic activity associated with C5a and/or preventing the assembly of the membrane attack complex associated with C5b.

Also useful in the present methods are naturally occurring or soluble forms of complement inhibitory compounds such as CR1, LEX-CRI, MCP, DAF, CD59, Factor H, cobra venom factor, FUT-175, complestatin, and K76 COOH. Other compounds which may be utilized to bind to or otherwise block the generation and/or activity of any of the human complement components include, but are not limited to, proteins, protein fragments, peptides, small molecules, RNA aptamers including ARC187 (which is commercially available from Archemix Corp., Cambridge, Mass.), L-RNA aptamers, spiegelmers, antisense compounds, serine protease inhibitors, molecules which may be utilized in RNA interference (RNAi) such as double stranded RNA including small interfering RNA (siRNA), locked nucleic acid (LNA) inhibitors, peptide nucleic acid (PNA) inhibitors, etc.

Functionally, one suitable class of compounds inhibits the cleavage of C5, which blocks the generation of potent proinflammatory molecules C5a and C5b-9 (terminal complement complex). Preferably, the compound does not prevent the formation of C3b, which subserves critical immunoprotective functions of opsonization and immune complex clearance.

While preventing the generation of these membrane attack complex molecules, inhibition of the complement cascade at C5 preserves the ability to generate C3b, which is critical for opsonization of many pathogenic microorganisms, as well as for immune complex solubilization and clearance. Retaining the capacity to generate C3b appears to be particularly important as a therapeutic factor in complement inhibition for hemolytic diseases, where increased susceptibility to thrombosis, infection, fatigue, lethargy and impaired clearance of immune complexes are pre-existing clinical features of the disease process.

Particularly useful compounds for use herein are antibodies that reduce, directly or indirectly, the conversion of complement component C5 into complement components C5a and C5b. One class of useful antibodies are those having at least one antigen binding site and exhibiting specific binding to human complement component C5. Particularly useful complement inhibitors are compounds which reduce the generation of C5a and/or C5b-9 by greater than about 30%. Anti-C5 antibodies that have the desirable ability to block the generation of C5a have been known in the art since at least 1982 (Moongkarndi et al., Immunobiol. 1982, 162:397; Moongkarndi et al., Immunobiol. 1983, 165:323). Antibodies known in the art that are immunoreactive against C5 or C5 fragments include antibodies against the C5 beta chain (Moongkarndi et al., Immunobiol. 1982, 162:397; Moongkarndi et al., Immunobiol. 1983, 165:323; Wurzner et al., 1991, supra; Mollnes et al., Scand. J. Immunol. 1988, 28:307-312); C5a (see for example, Ames et al., J. Immunol. 1994, 152:4572-4581, U.S. Pat. No. 4,686,100, and European patent publication No. 0 411 306); and antibodies against non-human C5 (see for example, Giclas et al., J. Immunol. Meth. 1987, 105:201-209). Particularly useful anti-C5 antibodies are h5G1.1-mAb, h5G1.1-scFv and other functional fragments of h5G1.1. Methods for the preparation of h5G1.1-mAb, h5G1.1-scFv and other functional fragments of h5G1.1 are described in U.S. Pat. No. 6,355,245 and “Inhibition of Complement Activity by Humanized Anti-C5 Antibody and Single Chain Fv”, Thomas et al., Molecular Immunology, Vol. 33, No. 17/18, pages 1389-1401, 1996, the disclosures of which are incorporated herein in their entirety by this reference. The antibody h5G1.1-mAb is currently undergoing clinical trials under the tradename eculizumab.

Hybridomas producing monoclonal antibodies reactive with complement component C5 can be obtained according to the teachings of Sims, et al., U.S. Pat. No. 5,135,916. Antibodies are prepared using purified components of the complement C5 component as immunogens according to known methods. In accordance with this disclosure, complement component C5, C5a or C5b is preferably used as the immunogen. In accordance with particularly preferred useful embodiments, the immunogen is the alpha chain of C5.

Particularly useful antibodies share the required functional properties discussed in the preceding paragraph and have any of the following characteristics: (1) they compete for binding to portions of C5 that are specifically immunoreactive with 5G1I.1; (2) they specifically bind to the C5 alpha chain—such specific binding, and competition for binding can be determined by various methods well known in the art, including the plasmon surface resonance method (Johne et al., J. Immunol. Meth. 1993, 160:191-198); and (3) they block the binding of C5 to either C3 or C4 (which are components of the C5 convertases).

The compound that inhibits the production and/or activity of at least one complement component can be administered in a variety of unit dosage forms. The dose will vary according to the particular compound employed. For example, different antibodies may have different masses and/or affinities, and thus require different dosage levels. Antibodies prepared as fragments (e.g., Fab, F(ab′)₂, scFv) will also require differing dosages than the equivalent intact immunoglobulins, as they are of considerably smaller mass than intact immunoglobulins, and thus require lower dosages to reach the same molar levels in the patient's blood.

The dose will also vary depending on the manner of administration, the particular symptoms of the patient being treated, the overall health, condition, size, and age of the patient, and the judgment of the prescribing physician.

Administration of the compound that inhibits the production and/or activity of at least one complement component will preferably be via intravenous infusion by injection but may be in an aerosol form with a suitable pharmaceutical carrier, subcutaneous injection, orally, or sublingually. Other routes of administration may be used if desired.

It is further contemplated that a combination therapy can be used wherein a complement-inhibiting compound is administered in combination with a regimen of known therapy for hemolytic disease. Such regimens include administration of 1) one or more compounds known to increase hematopoiesis (for example, either by boosting production, eliminating stem cell destruction or eliminating stem cell inhibition) in combination with 2) a compound selected from the group consisting of compounds which bind to one or more complement components, compounds which block the generation of one or more complement components and compounds which block the activity of one or more complement components. Suitable compounds known to increase hematopoiesis include, for example, steroids, immunosuppressants (such as, cyclosporin), anti-coagulants (such as, warfarin), folic acid, iron and the like, erythropoietin (EPO), antithymocyte globulin (ATG), antilymphocyte globulin (ALG), EPO derivatives, EPO mimetics, and darbepoetin alfa (commercially available as Aranesp® from Amgen, Inc., Thousand Oaks, Calif. (Aranesp® is a man-made form of EPO produced in Chinese hamster ovary (CHO) cells by recombinant DNA technology)). In particularly useful embodiments, erythropoietin (EPO) (a compound known to increase hematopoiesis), EPO derivatives, or darbepoetin alfa may be administered in combination with an anti-C5 antibody selected from the group consisting of h5G1.I-mAb, h5G1.1-scFv and other functional fragments of h5G1.1.

Formulations suitable for injection are found in Remington's Pharmaceutical Sciences, Mack Publishing Company, Philadelphia, Pa., 17th ed. (1985). Such formulations must be sterile and non-pyrogenic, and generally will include a pharmaceutically effective carrier, such as saline, buffered (e.g., phosphate buffered) saline, Hank's solution, Ringer's solution, dextrose/saline, glucose solutions, and the like. The formulations may contain pharmaceutically acceptable auxiliary substances as required, such as, tonicity adjusting agents, wetting agents, bactericidal agents, preservatives, stabilizers, and the like.

The present disclosure contemplates methods of reducing hemolysis in a patient afflicted with a hemolytic disease by administering one or more compounds which bind to or otherwise block the generation and/or activity of one or more complement components. Reducing hemolysis means that the duration of time a person suffers from hemolysis is reduced by about 25% or more. The effectiveness of the treatment can be evaluated in any of the various manners known to those skilled in the art for determining the level of hemolysis in a patient. One qualitative method for detecting hemolysis is to observe the occurrences of hemoglobinuria. Quite surprisingly, treatment in accordance with the present methods reduces hemolysis as determined by a rapid reduction in hemoglobinunra.

A more qualitative manner of measuring hemolysis is to measure lactate dehydrogenase (LDH) levels in the patient's bloodstream. LDH catalyzes the interconversion of pyruvate and lactate. Red blood cells metabolize glucose to lactate, which is released into the blood and is taken up by the liver. LDH levels are used as an objective indicator of hemolysis. As those skilled in the art will appreciate, measurements of “upper limit of normal” levels of LDH will vary from lab to lab depending on a number of factors including the particular assay employed and the precise manner in which the assay is conducted. Generally speaking, however, the present methods can reduce hemolysis in a patient afflicted with a hemolytic disease as reflected by a reduction of LDH levels in the patients to within 20% of the upper limit of normal LDH levels. Alternatively, the present methods can reduce hemolysis in a patient afflicted with a hemolytic disease as reflected by a reduction of LDH levels in the patients of greater than 50% of the patient's pre-treatment LDH level, preferably greater than 65% of the patient's pre-treatment LDH level, most preferably greater than 80% of the patient's pre-treatment LDH level.

Another quantitative measurement of a reduction in hemolysis is the presence of GPI-deficient red blood cells (PNH red blood cells). As those skilled in the art will appreciate, PNH red blood cells have no GPI-anchor protein expression on the cell surface. The proportion of GPI-deficient cells (PNH cells) can be determined by flow cytometry using, for example, the technique described in Richards, et al., Clin. Appl. Immunol. Rev., vol. 1, pages 315-330, 2001. The absolute number of the PNH cells can then be determined. The present methods can reduce hemolysis in a patient afflicted with a hemolytic disease as reflected by an increase in PNH red blood cells. Preferably an increase in PNH red blood cell levels in the patient of greater than 25% of the total red blood cell count is achieved, more preferably an increase in PNH red blood cell levels in the patients greater than 50% of the total red blood cell count is achieved, most preferably an increase in PNH red blood cell levels in the patients greater than 75% of the total red blood cell count is achieved.

Methods of reducing one or more symptoms associated with PNH or other hemolytic diseases are also within the scope of the present disclosure. Such symptoms include, for example, abdominal pain, fatigue, and dyspnea. Symptoms can be the direct result of lysis of red blood cells (e.g., hemoglobinuria, anemia, fatigue, low red blood cell count, etc.) or the symptoms can result from low nitric oxide (NO) levels in the patient's bloodstream (e.g., abdominal pain, erectile dysfunction, dysphagia, thrombosis, etc.). It has recently been reported that patients with greater than 40% PNH granulocyte clone have an increased incidence of thrombosis, abdominal pain, erectile dysfunction and dysphagia, indicating a high hemolytic rate (see Moyo et al., British J. Haematol. 126:133-138 (2004)).

In particularly useful embodiments, the present methods provide a reduction in one or more symptoms associated with PNH or other hemolytic diseases in a patient having a platelet count in excess of 30,000 per microliter (a hypoplastic patient), preferably in excess of 75,000 per microliter, most preferably in excess of 150,000 per microliter. In other embodiments, the present methods provide a reduction in one or more symptoms associated with PNH or other hemolytic diseases in a patient where the proportion of PNH type III red blood cells of the subject's total red blood cell content is greater than 10%, preferably greater than 25%, most preferably in excess of 50%. In yet other embodiments, the present methods provide a reduction in one or more symptoms associated with PNH or other hemolytic diseases in a patient having a reticulocyte count in excess of 80×10⁹per liter, more preferably in excess of 120×10⁹ per liter, most preferably in excess of 150×10⁹ per liter. Patients in the most preferable ranges recited above have active bone marrow and will produce adequate numbers of red blood cells. While in a patient afflicted with PNH or other hemolytic disease the red blood cells may be defective in one or more ways (e.g., GPI deficient), the present methods are particularly useful in protecting such cells from lysis resulting from complement activation. Thus, patients within the preferred ranges benefit most from the present methods.

In one aspect, a method of reducing fatigue is contemplated, the method including the step of administering to a subject having or susceptible to a hemolytic disease a compound which binds to or otherwise blocks the generation and/or activity of one or more complement components. Reducing fatigue means the duration of time a person suffers from fatigue is reduced by about 25% or more. Fatigue is a symptom believed to be associated with intravascular hemolysis as the fatigue relents when hemoglobinuria resolves even when the anemia persists. By reducing the lysis of red blood cells, the present methods reduce fatigue. Patients within the above-mentioned preferred ranges of type III red blood cells, reticulocytes and platelets benefit most from the present methods.

In another aspect, a method of reducing abdominal pain is contemplated, the method including the step of administering to a subject having or susceptible to a hemolytic disease a compound which binds to or otherwise blocks the generation and/or activity of one or more complement components. Reducing abdominal pain means the duration of time a person suffers from abdominal pain is reduced by about 25% or more. Abdominal pain is a symptom resulting from the inability of a patient's natural levels of haptoglobin to process all the free hemoglobin released into the bloodstream as a result of intravascular hemolysis, resulting in the scavenging of NO and intestinal dystonia and spasms. By reducing the lysis of red blood cells, the present methods reduce the amount of free hemoglobin in the bloodstream, thereby reducing abdominal pain. Patients within the above-mentioned preferred ranges of type III red blood cells, reticulocytes and platelets benefit most from the present methods.

In another aspect, a method of reducing dysphagia is contemplated, the method including the step of administering to a subject having or susceptible to a hemolytic disease a compound which binds to or otherwise blocks the generation and/or activity of one or more complement components. Reducing dysphagia means the duration of time a person has dysphagia attacks is reduced by about 25% or more. Dysphagia is a symptom resulting from the inability of a patient's natural levels of haptoglobin to process all the free hemoglobin released into the bloodstream as a result of intravascular hemolysis, resulting in the scavenging of NO and esophageal spasms. By reducing the lysis of red blood cells, the present methods reduce the amount of free hemoglobin in the bloodstream, thereby reducing dysphagia. Patients within the above-mentioned preferred ranges of type III red blood cells, reticulocytes and platelets benefit most from the present methods.

In yet another aspect, a method of reducing erectile dysfuinction is contemplated, the method including the step of administering to a subject having or susceptible to a hemolytic disease a compound which binds to or otherwise blocks the generation and/or activity of one or more complement components. Reducing erectile dysfunction means the duration of time a person suffers from erectile dysfunction is reduced by about 25% or more. Erectile dysfunction is a symptom believed to be associated with scavenging of NO by free hemoglobin released into the bloodstream as a result of intravascular hemolysis. By reducing the lysis of red blood cells, the present methods reduce the amount of free hemoglobin in the bloodstream, thereby increasing serum levels of NO and reducing erectile dysfunction. Patients within the above-mentioned preferred ranges of type III red blood cells, reticulocytes and platelets benefit most from the present methods.

In yet another aspect, a method of reducing hemoglobinuria is contemplated, the method including the step of administering to a subject having or susceptible to a hemolytic disease a compound which binds to or otherwise blocks the generation and/or activity of one or more complement components. Reducing hemoglobinuria means a reduction in the number of times a person has red, brown, or darker urine, wherein the reduction is typically about 25% or more. Hemoglobinuria is a symptom resulting from the inability of a patient's natural levels of haptoglobin to process all the free hemoglobin released into the bloodstream as a result of intravascular hemolysis. By reducing the lysis of red blood cells, the present methods reduce the amount of free hemoglobin in the bloodstream and urine thereby reducing hemoglobinuria. Quite surprisingly, the reduction in hemoglobinuria occurs rapidly. Patients within the above-mentioned preferred ranges of type III red blood cells, reticulocytes and platelets benefit most from the present methods.

In still another aspect, a method of reducing thrombosis is contemplated, the method including the step of administering to a subject having or susceptible to a hemolytic disease a compound which binds to or otherwise blocks the generation and/or activity of one or more complement components. Reducing thrombosis means the duration of time a person has thrombosis attacks is reduced by about 25% or more or that the frequency of thrombosis attacks is reduced by about 25% or more over a period of one or more years. Thrombosis is a symptom believed to be associated with scavenging of NO by free hemoglobin released into the bloodstream as a result of intravascular hemolysis. By reducing the lysis of red blood cells, the present methods reduce the amount of free hemoglobin in the bloodstream, thereby increasing serum levels of NO and reducing thrombosis.

Thrombosis is thought to be multi-factorial in etiology including NO scavenging by free hemoglobin, exposure of prothrombotic surfaces from lysed red blood cell membranes, and changes in the endothelium surface by cell free heme. The intravascular release of free hemoglobin may directly contribute to small vessel thrombosis. NO has been shown to inhibit platelet aggregation, induce disaggregation of aggregated platelets and inhibit platelet adhesion. Conversely, NO scavenging by hemoglobin or the reduction of NO generation by the inhibition of arginine metabolism results in an increase in platelet aggregation. By reducing the lysis of red blood cells, the present methods reduce the amount of free hemoglobin in the bloodstream, thereby increasing serum levels of NO and reducing thrombosis.

In particularly useful embodiments, the present methods reduce thrombosis, especially in patients having a platelet count in excess of 30,000 per microliter, preferably in excess of 75,000 per microliter, most preferably in excess of 150,000 per microliter. In other embodiments, the present methods reduce thrombosis in patients where the proportion of PNH type III red blood cells of the subject's total red blood cell content is greater than 1%, preferably greater than 10%, more preferably greater than 25%, even more preferably in excess of 50%, and most preferably in excess of 75% (see, e.g., Hall et al., Blood 102:3587-3591 (2003); Audebert et al., J. Neurol. 252:1379-1386 (2005)). In yet other embodiments, the present methods reduce transfusion thrombosis in patients having a reticulocyte count in excess of 80×10⁹ per liter, more preferably in excess of 120×10⁹ per liter, most preferably in excess of 150×10⁹ per liter.

In still another aspect, a method of reducing anemia is contemplated, the method including the step of administering to a subject having or susceptible to a hemolytic disease a compound which binds to or otherwise blocks the generation and/or activity of one or more complement components. Reducing anemia means the duration of time a person has anemia is reduced by about 25% or more. Anemia in hemolytic diseases results from the blood's reduced capacity to carry oxygen due to the loss of red blood cell mass. By reducing the lysis of red blood cells, the present methods assist red blood cell levels to increase thereby reducing anemia.

In another aspect, a method of increasing the total endogenous red blood cell count in a patient afflicted with a hemolytic disease is contemplated. By increasing the patient's RBC count, fatigue, anemia and the patient's need for blood transfusions is reduced. The reduction in transfusions can be in frequency of transfusions, amount of blood units transfused, or both.

The method of increasing red blood cell count in a patient afflicted with a hemolytic disease includes the step of administering a compound which binds to or otherwise blocks the generation and/or activity of one or more complement components to a patient afflicted with a hemolytic disease. In particularly useful embodiments, the present methods increase red blood cell count in a patient afflicted with a hemolytic disease, especially patients having a platelet count in excess of 30,000 per microliter, preferably in excess of 75,000 per microliter, most preferably in excess of 150,000 per microliter. In other embodiments, the present methods increase red blood cell count in a patient afflicted with a hemolytic disease where the proportion of PNH type III red blood cells of the subject's total red blood cell content is greater than 1%, preferably greater than 10%, more preferably greater than 25%, even more preferably in excess of 50%, and most preferably in excess of 75%. In yet other embodiments, the present methods increase red blood cell count in a patient afflicted with a hemolytic disease having a reticulocyte count in excess of 80×10⁹ per liter, more preferably in excess of 120×10⁹ per liter, most preferably in excess of 150×10⁹ per liter. In some embodiments, the methods of the present disclosure may result in a decrease in the frequency of transfusions by about 50%, typically a decrease in the frequency of transfusions by about 70%, more typically a decrease in the frequency of transfusions by about 90%.

In yet another aspect, the present disclosure contemplates a method of rendering a subject afflicted with a hemolytic disease less dependent on transfusions or transfusion-independent by administering a compound to the subject, the compound being selected from the group consisting of compounds which bind to one or more complement components, compounds which block the generation of one or more complement components and compounds which block the activity of one or more complement components. As those skilled in the art will appreciate, the normal life cycle for a red blood cell is about 120 days. Treatment for six months or more is required for the evaluation of transfusion independence given the long half life of red blood cells. It has unexpectedly been found that in some patients transfusion-independence can be maintained for twelve months or more, in some cases more than four years, long beyond the 120 day life cycle of red blood cells. In particularly useful embodiments, the present methods provide decreased dependence on transfusions or transfusion-independence in a patient afflicted with a hemolytic disease, especially patients having a platelet count in excess of 30,000 per microliter, preferably in excess of 75,000 per microliter, most preferably in excess of 150,000 per microliter. In other embodiments, the present methods provide decreased dependence on transfusions or transfusion-independence in a patient afflicted with a hemolytic disease where the proportion of PNH type III red blood cells of the subject's total red blood cell content is greater than 1%, preferably greater than 10%, more preferably greater than 25%, even more preferably in excess of 50%, and most preferably in excess of 75%. In yet other embodiments, the present methods provide decreased dependence on transfusions or transfusion-independence in a patient afflicted with a hemolytic disease having a reticulocyte count in excess of 80×10⁹ per liter, more preferably in excess of 120×10⁹ per liter, most preferably in excess of 150×10⁹ per liter.

Methods of increasing the nitric oxide (NO) levels in a patient having PNH or some other hemolytic disease are also within the scope of the present disclosure. These methods of increasing NO levels include the step of administering to a subject having or susceptible to a hemolytic disease a compound which binds to or otherwise blocks the generation and/or activity of one or more complement components. Low NO levels arise in patients afflicted with PNH or other hemolytic diseases as a result of scavenging of NO by free hemoglobin released into the bloodstream as a result of intravascular hemolysis. By reducing the lysis of red blood cells, the present methods reduce the amount of free hemoglobin in the bloodstream, thereby increasing serum levels of NO. In particularly useful embodiments, NO homeostasis is restored as evidenced by a resolution of symptoms attributable to NO deficiencies.

Without intending to limit it in any manner, the present application will be more fully described by the following examples.

### EXAMPLES

### Example 1

Eleven patients participated in therapy trials to evaluate the effects of anti-C5 antibody on PNH and symptoms associated therewith. PNH patients were transfusion-dependent and hemolytic. Patients were defined as transfusion dependent with a history of four or more transfusions within twelve months. The median number of transfusions within the patient pool was nine in the previous twelve months. The median number of transfusion units used in the previous twelve months was twenty-two for the patient pool.

Over the course of four weeks, each of 11 patients received a weekly 600 mg intravenous infusion of anti-C5 antibody for approximately thirty minutes. The specific anti-C5 antibody used in the study was eculizumab. Patients received 900 mg of eculizumab 1 week later and then 900 mg on a biweekly basis. The first twelve weeks of the study constituted the pilot study. Following completion of the initial acute phase twelve week study, all patients participated in an extension study conducted to a total of 64 weeks. Ten of the eleven patients participated in an extension study conducted to a total of two years.

The effect of anti-C5 antibody treatments on PNH type III red blood cells (“RBCs”) was tested. “PNH Type” refers to the density of GPI-anchored proteins expressed on the cell surface. Type I is normal expression, Type II is intermediate expression, and Type III has no GPI-anchor protein expression on the cell surface. The proportion of GPI-deficient cells is determined by flow cytometry in the manner described in Richards, et al., Clin. Appl. Immunol. Rev., vol. 1, pages 315-330, 2001. As compared to pre-therapy conditions, PNH Type III red blood cells increased more than 50% during the extension study. The increase from a pre-study mean value of 36.7% of all red blood cells to a 64 week mean value of 58.4% of Type III red blood cells indicated that hemolysis had decreased sharply. See Table 1, below. Eculizumab therapy protected PNH type III RBCs from complement-mediated lysis, prolonging the cells' survival. This protection of the PNH-affected cells reduced the need for transfusions, paroxysms and overall hemolysis in all patients in the trial.

TABLE 1
PNH Cell Populations Pre- and Post-Eculizumab Treatment
in All Patients

| | Proportion of PNH Cells (%) | | | |
|---|---|---|---|---|
| PNH Cell Type | baseline | 12 weeks | 64 weeks | p-value^{a} |
| Type III RBCs | 36.7 +/− 5.9 | 59.2 +/− 8.0 | 58.4 +/− 8.5 | 0.005 |
| Type II RBCs | 5.3 +/− 1.4 | 7.5 +/− 2.1 | 13.2 +/− 2.4 | 0.013 |
| Type III WBCs | 92.1 +/− 4.6 | 89.9 +/− 6.6 | 91.1 +/− 5.8 | N.S. |
| Type III | 92.4 +/− 2.4 | 93.3 +/− 2.8 | 92.8 +/− 2.6 | N.S. |
| Platelets | | | | |

^{a}comparison of mean change from baseline to 64 weeks

During the course of the two year extension study, it was found that PNH red cells with a complete deficiency of GPI-linked proteins (Type III red cells) progressively increased during the treatment period from a mean of 36.7% to 58.9% (p=0.001) while partially deficient PNH red cells (Type II) increased from 5.3% to 8.7% (p=0.01). There was no concomitant change in the proportion of PNH neutrophils in any of the patients during eculizumab therapy, indicating that the increase in the proportion of PNH red cells was due to a reduction in hemolysis and transfusions rather than a change in the PNH clone(s) themselves.

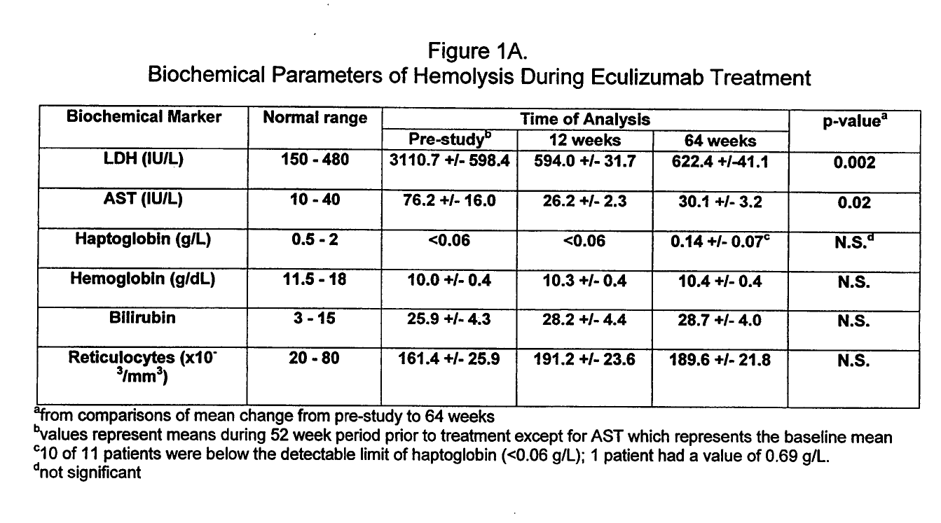

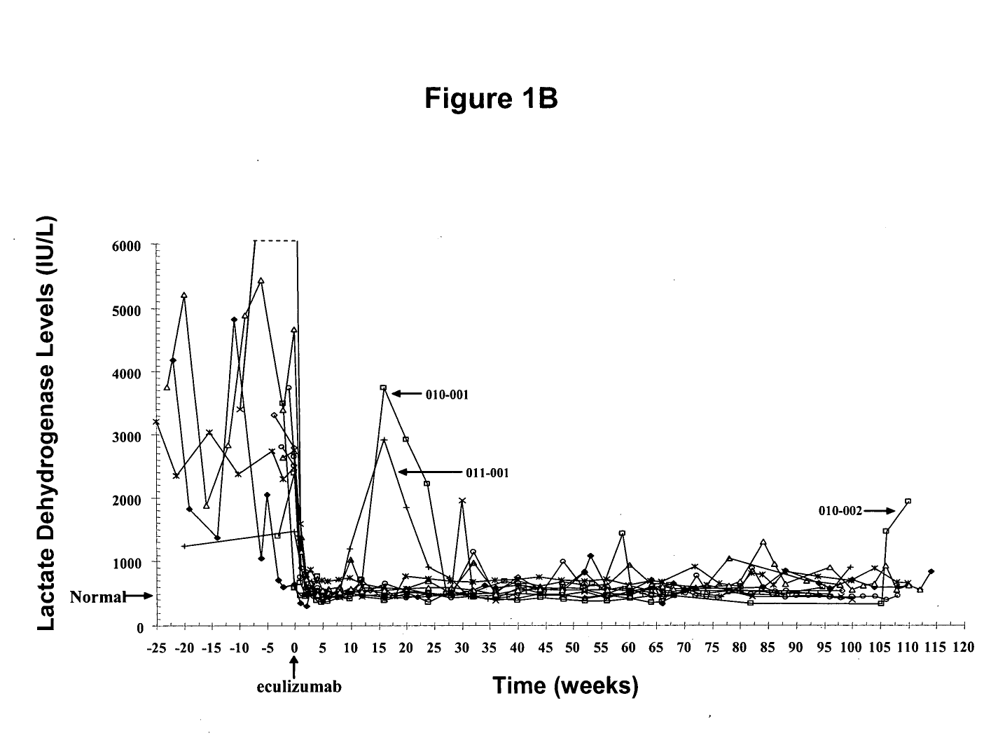

The effect of anti-C5 antibody treatments on lactate dehydrogenase levels (“LDH”) was measured on all eleven patients. LDH catalyzes the interconversion of pyruvate and lactate. Red blood cells metabolize glucose to lactate, which is released into the blood and is taken up by the liver. LDH levels are used as an objective indicator of hemolysis. The LDH levels were decreased by greater than 80% as compared to pre-treatment levels. The LDH levels were lowered from a pre-study mean value of 3111 U/L to a mean value of 594 U/L during the pilot study and a mean value of 622 U/L after 64 weeks (p=0.002 for 64 week comparison; see FIGS. 1A and 1B).

Similarly, aspartate aminotransferase (AST) levels, another marker of red blood cell hemolysis, decreased from a mean baseline value of 76 IU/L to 26 IU/L and 30 IU/L during the 12 and 64 weeks of treatment, respectively (p=0.02 for 64 week comparison). Levels of haptoglobin, hemoglobin and bilirubin, and numbers of reticulocytes, did not change significantly from prestudy values during the 64 week treatment period.

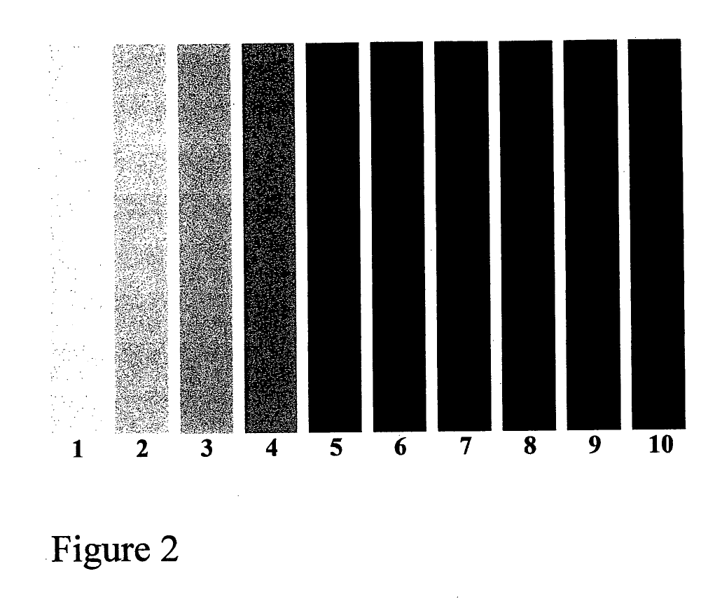

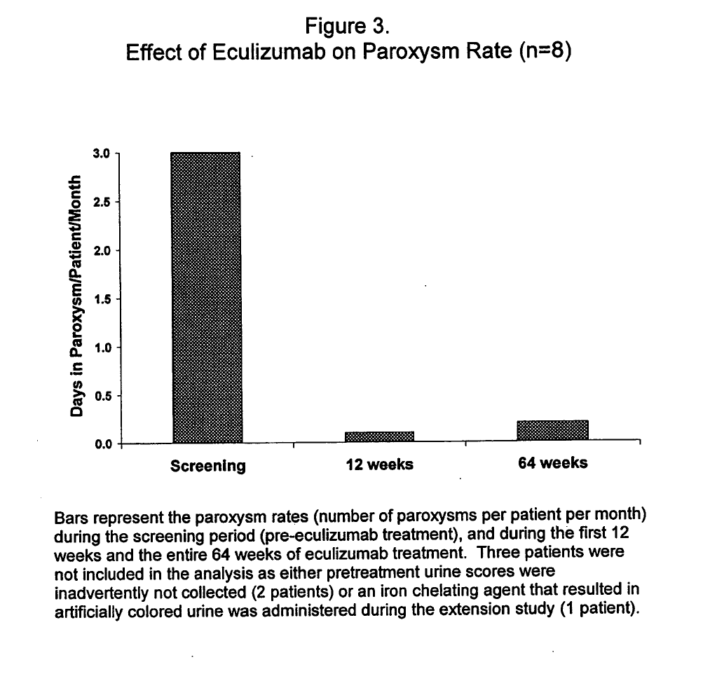

Paroxysm rates were measured and compared to pre-treatment levels. Paroxysm as used in this disclosure is defined as incidences of dark-colored urine with a colorimetric level of 6 of more on a scale of 1-10. FIG. 2 shows the urine color scale devised to monitor the incidence of paroxysm of hemoglobinuria in patients with PNH before and during treatment. As compared to pre-treatment levels, the paroxysm percentage rate was reduced by 93% (see, FIG. 3) from 3.0 paroxysms per patient per month before eculizumab treatment to 0.1 paroxysm per patient per month during the initial 12 weeks and 0.2 paroxysm per patient per month during the 64 week treatment (FIG. 3 (p<0.001)).

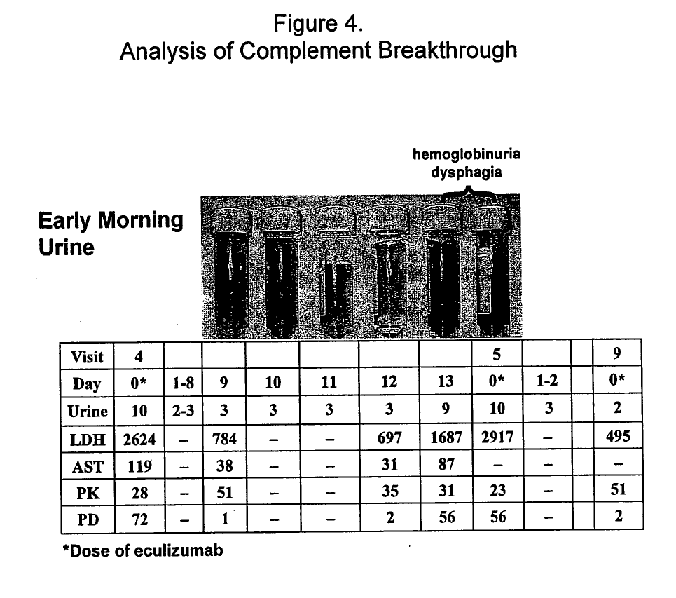

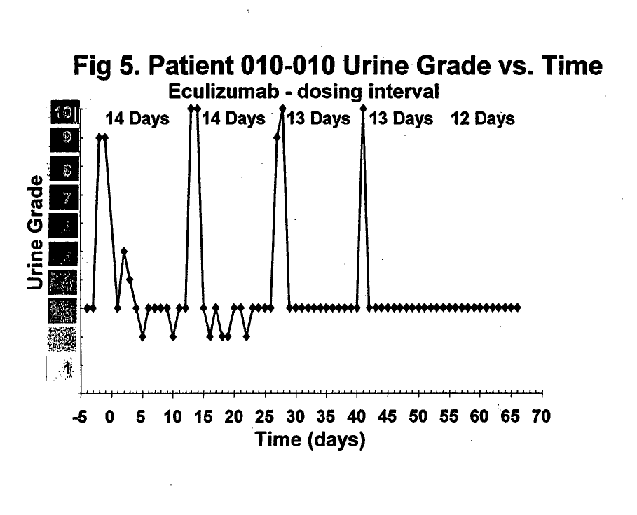

Serum hemolytic activity in nine of the eleven patients was completely blocked throughout the 64 week treatment period with trough levels of eculizumab at equilibrium ranging from approximately 35 μg/mL to 350 μg/mL. During the extension study, 2 patients did not sustain levels of eculizumab necessary to consistently block complement. This breakthrough in serum hemolytic activity occurred in the last 2 days of the 14 day dosing interval, a pattern that was repeated between multiple doses. In one of the patients, as seen in FIG. 4, break-through of complement blockade resulted in hemoglobinuria, dysphagia, and increased LDH and AST, which correlated with the return of serum hemolytic activity. At the next dose, symptoms resolved (FIG. 5) and reduction in the dosing interval from 900 mg every 14 days to 900 mg every 12 days resulted in a regain of complement control which was maintained over the extension study to 64 weeks in both patients. This patient showed a 24 hour resolution of dysphagia and hemoglobinuria. A reduction in the dosing interval from 14 to 12 days was sufficient to maintain levels of eculizumab above 35 μg/mL and effectively and consistently blocked serum hemolytic activity for the remainder of the extension study for both patients.

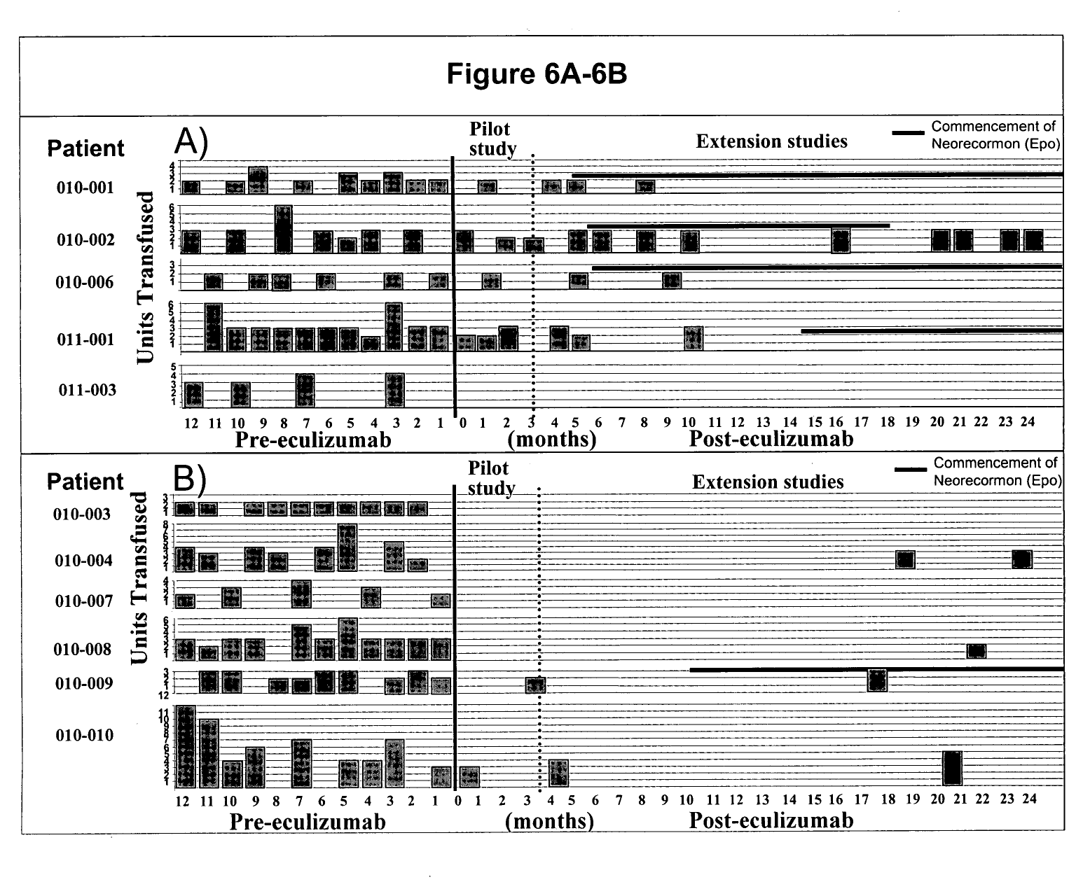

The patients' need for transfusions was also reduced by the treatment with eculizumab. FIG. 6a compares the number of transfusion units required per patient per month, prior to and during treatment with an anti-C5 antibody for cytopenic patients, while FIG. 6b compares the number of transfusion units required per patient per month, prior to and during treatment with an anti-C5 antibody for non-cytopenic patients. A significant reduction in the need for transfusion was also noted in the entire group (mean transfusion rates decreased from 2.1 units per patient per month during a 1 year period prior to treatment to 0.6 units per patient per month during the initial 12 weeks and 0.5 units per patient per month during the combined 64 week treatment period), with non-cytopenic patients benefiting the most. In fact, four of the non-thrombocytopenic patients with normal platelet counts (≧150,000 per microliter) became transfusion-independent during the 64 week treatment.

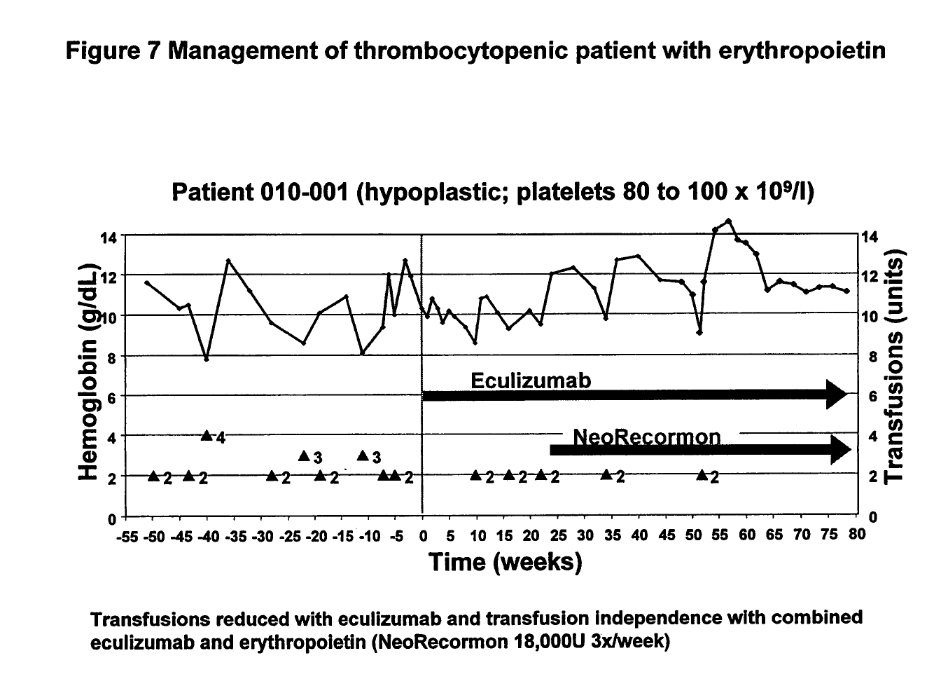

The effect of eculizumab administered in combination with erythropoietin (EPO) was also evaluated in a thrombocytopenic patient. EPO (NeoRecormon™, Roche Pharmaceuticals, Basel, Switzerland) was administered in an amount of 18,000 I.U. three times per week beginning in week 23 of the study. As shown in FIG. 7, the frequency of transfusions required for this patient was significantly reduced, and soon halted.

For the two year extension study, 10 of the 11 patients from the initial 3 month study continued to receive 900 mg of eculizumab every other week. (One patient discontinued eculizumab therapy after 23 months.) Six of the 11 patients had normal platelet counts (no clinical evidence of marrow failure) whereas 5 of the 11 had low platelet counts. For the patient who discontinued eculizumab therapy after 23 months, intravascular hemolysis was successfully controlled by eculizumab, but the patient continued to be transfused even after erythropoietin therapy. This patient had the most severe hypoplasia at the start of eculizumab therapy with a platelet count below 30×10⁹/L, suggesting that the ongoing transfusions were likely a result of the underlying bone marrow failure.

Results of the two year extension study also demonstrated that there was a statistically significant decrease in transfusion requirements for the patients. Three patients remained transfusion independent during the entire two year treatment period, and four cytopenic patients became transfusion independent, three following treatment with EPO (NeoRecormon™). The reduction in transfusion requirements was found to be most pronounced in patients with a good marrow reserve.

Pharmacodynamic levels were measured and recorded according to eculizumab doses. The pharm acodynamic analysis of eculizumab was determined by measuring the capacity of patient serum samples to lyse chicken erythrocytes in a standard total human serum complement hemolytic assay. Briefly, patient samples or human control serum (Quidel, San Diego, Calif.) was diluted to 40% vol/vol with gelatin veronal-buffered saline (GVB2+, Advanced Research technologies, San Diego, Calif.) and added in triplicate to a 96-well plate such that the final concentration of serum in each well was 20%. The plate was then incubated at room temperature while chicken erythrocytes (Lampire Biologics, Malvern, Pa.) were washed. The chicken erythrocytes were sensitized by the addition of anti-chicken red blood cell polyclonal antibody (0.1% vol/vol). The cells were then washed and resuspended in GVB2+ buffer. Chicken erythrocytes (2.5×10⁶cells/30 μL) were added to the plate containing human control serum or patient samples and incubated at 37° C. for 30 min. Each plate contained six additional wells of identically prepared chicken erythrocytes of which four wells were incubated with 20% serum containing 2 mM EDTA as the blank and two wells were incubated with GVB2+buffer alone as a negative control for spontaneous hemolysis. The plate was then centrifuged and the supernatant transferred to a new flat bottom 96-well plate. Hemoglobin release was determined at OD 415 nm using a microplate reader. The percent hemolysis was determined using the following formula: Percent Hemolysis=100×((OD patient sample−OD blank)/(OD human serum control−OD blank )).

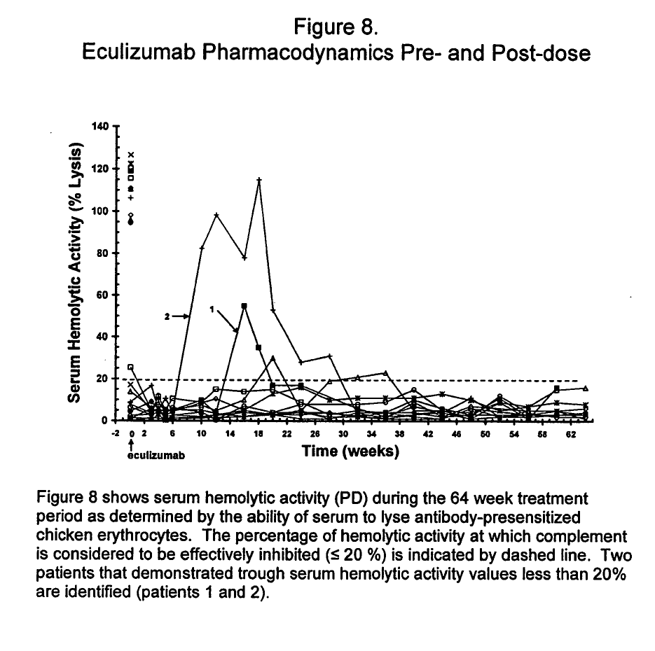

The graph of the pharmacodynamics (FIG. 8), the study of the physiological effects, shows the percentage of serum hemolytic activity (i.e. the percentage of cell lysis) over time. Cell lysis was dramatically reduced in the majority of the patients to below 20% of normal serum hemolytic activity while under eculizumab treatment. Two patients exhibited a breakthrough in hemolytic activity, but complement blockade was permanently restored by reducing the dosing interval to 12 days (See, FIG. 4).

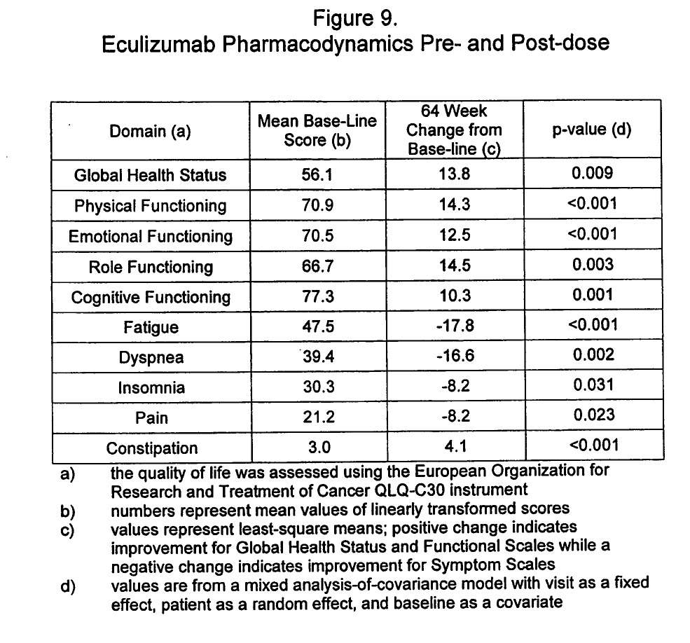

Improvement of quality of life issues was also evaluated using the European Organization for Research and Treatment of Cancer Core (http://www.eortc.be) questionnaires (“EORTC QLC-C30”). Each of the participating patients completed the QLC-30 questionnaire before and during the eculizumab therapy. Overall improvements were observed in global health status, physical functioning, role functioning, emotional functioning, cognitive functioning, fatigue, pain, dyspnea and insomnia. (See FIG. 9).

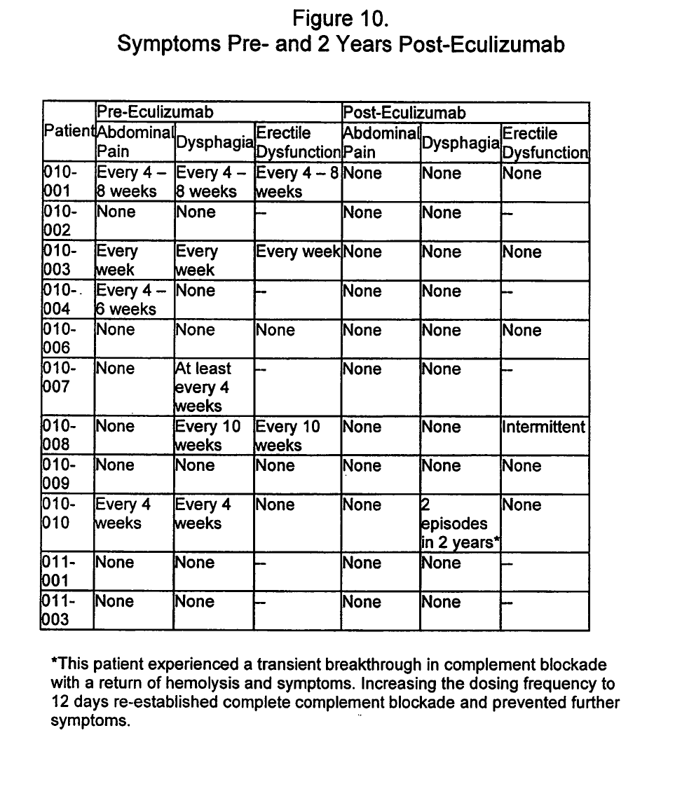

Patients in the two year study experienced a reduction in adverse symptoms associated with PNH. For example, as set forth in FIG. 10, there was a demonstrated decrease of abdominal pain, dysphagia, and erectile dysfunction after administration of eculizumab in those patients reporting those symptoms before administration of eculizumab.

### Example 2

Description of Clinical Studies

The safety and efficacy of eculizumab was assessed in three separate studies including an 87 patient randomized, double-blind, placebo-controlled 26 week phase 3 study (Study C04-001), an ongoing 97 patient open-label 52 week phase 3 study (Study C04-002), and an 11 patient open-label 12 week phase 2 study (Study C02-001; this study had two study-specific extension studies [E02-001 and X03-001] totaling an additional 156 weeks). All patients successfully completing Studies C04-001, C04-002, or C02-001/E02-001/X03-001 were eligible to enroll in an ongoing open-label 104 week phase 3 extension study (Study E05-001) which is anticipated to enroll approximately 190 patients. The E05-001 study provides additional long-term safety and efficacy data of eculizumab in the overall population of PNH patients and includes the collection of thromboembolic event rates with eculizumab treatment across the pooled eculizumab treatment groups from the parent studies described above. The pre-specified secondary endpoint of thromboembolic event rate in Study E05-001 was designed to assess the thromboembolic event rate in each of the Study E05-001 patients before eculizumab treatment, during eculizumab treatment in each of the Study C04-001, C04-002, and C02-001/E02-001/X03-001 patients, and during eculizumab treatment in the overall patient population for all studies. Thrombotic event rates were captured as major adverse vascular events (MAVE, See Table 2).

In all studies, eculizumab-treated patients were administered 600 mg study drug every week for 4 weeks, 900 mg in week 5, and then a 900 mg dose every 14±2 days for the study duration.

In Study C04-001, C04-002, and C02-001/E02-001/X03-001, eculizumab treatment was associated with highly statistically and clinically significant improvements in all pre-specified primary and secondary endpoints.

TABLE 2
LIST of MAJOR ADVERSE VASCULAR EVENTS (MAVE)

| | |
|---|---|
| Thrombophlebitis/Deep Vein | Renal Vein Thrombosis |
| Thrombosis | |
| Pulmonary Embolus | Mesenteric Vein Thrombosis |
| Cerebrovascular Accident | Portal Vein Thrombosis (Budd-Chiari) |
| Amputation | Gangrene |
| Myocardial Infarction | Acute Peripheral Vascular Occlusion |
| Transient Ischemic Attack | Sudden Death |
| Unstable Angina | |

Effect of Eculizumab on the Pathophysiological Pathways Leading to Clinically Symptomatic Thrombosis

Thromboembolic (TE) events are frequently tied directly to intravascular hemolysis in PNH. Intravascular hemolysis leads to accumulation of free hemoglobin in the plasma which has been demonstrated to deplete nitric oxide and subsequently lead to thrombus formation.

Treatment with eculizumab markedly reduces intravascular hemolysis, as measured by a decrease in median LDH, from 2,042 U/L pre-treatment to 261 U/L at 26 weeks with eculizumab treatment in the combined C04-001 and C04-002 studies (P<0.00l).

Treatment with eculizumab markedly reduces circulating levels of cell-free hemoglobin, as measured by median free hemoglobin levels, from 36.7 mg/dL pre-treatment to 5.6 mg/dL at 26 weeks with eculizumab treatment in the combined C04-001 and C04-002 studies (P<0.001).

Treatment with eculizumab effectively reduces the consumption of nitric oxide, as measured by the median change in nitric oxide consumption, with median pre-treatment nitric oxide of 9.3 μM decreasing by 67.1% at week 26 with eculizumab treatment and with pre-treatment nitric oxide of 9.9 μM increasing by 14.9% at week 26 with placebo in the C04-001 study (P<0.001).

Effect of Eculizumab on Thrombotic Rate in Overall Study Population

Eculizumab-treatment TE events were determined for all patients that entered into and received eculizumab in the C04-001, C04-002, C02-001, E02-001, X03-001 and E05-001 PNH clinical studies on an intention-to-treat basis. TE events were defined by the MAVE criteria (see Table 2 above) in the C04-001, C04-002, and E05-001 studies (primary adverse event and medical history listings were used for the C02-001, E02-001 and X03-001 studies). Patient years of eculizumab exposure were calculated for the completed C04-001, C02-001, E02-001 and X03-001 studies. For the C04-002 study, patient years of eculizumab exposure were determined for each patient after 26 weeks of treatment (the 6 month interim analysis). In the E05-001 study, patient years of exposure was determined for all patients through April 2006.

The pre-treatment patient years was determined from the earlier of diagnosis of PNH or first thrombotic event prior to enrollment into the parent PNH clinical studies (C04-001, C04-002, C02-001) and also included patient years from placebo-treated patients in the C04-001 study. The total pre-eculizumab treatment TE events included all TE events in all patients prior to enrollment in C04-001, C04-002, and C02-001 plus the TE events during placebo treatment in the C04-001 study (i.e., total pre-eculizumab treatment period TE events equals the sum of pre-eculizumab treatment TE events in C04-001, C04-002, and C02-001 in Table 3 plus the pre-C04-001 TE events in Table 4 plus the placebo-treatment TE events in Table 4). The total eculizumab period TE events included all TE events during the period commencing from the first eculizumab dose. The primary TE analysis (i.e., E05-001 secondary endpoint) was performed with a signed rank test.

Compared to the rate of thromboembolic events before treatment, eculizumab treatment resulted in a reduction in the TE event rate in the same patients in each of the individual clinical studies and a significant reduction in the TE event rate overall. The overall TE event rate was reduced from 7.49 TE events per 100 patient years pre-eculizumab treatment to 1.22 TE events per 100 patient years in the same patients with eculizumab treatment (P<0.001). This represented a relative reduction of 84% and an absolute reduction of 6.27 TE events per 100 patient years. Thromboembolic event rates are shown in Table 3.

TABLE 3
Overall Thromboembolic Events in Patients Prior to Start of Eculizumab
Treatment and During Eculizumab Treatment in C04-001, C04-002,
C02-001/E02-001/X03-001 and E05-001

| | | | C02-001/ | E05-001 |
|---|---|---|---|---|
| | | C04- | E02-001/ | (All studies |
| | C04-001 | 002 | X03-001 | combined) |
| Pre-Treatment | | | | |
| Patients (n) | 43 | 97 | 11 | 195 |
| MAVE Events (n) | 16 | 93 | 5 | 126 |
| Patient Years (n) | 309.0 | 718.3 | 161.7 | 1683.4 |
| MAVE Event Rate | 5.18 | 12.95 | 3.09 | 7.49 |
| (n per 100 patient years) | | | | |
| Eculizumab Treatment | | | | |
| Patients (n) | 43 | 97 | 11 | 195 |
| MAVE Events (n) | 0 | 2 | 0 | 2 |
| Patient Years (n) | 21.8 | 48.2 | 35.8 | 164.1 |
| MAVE Event Rate | 0.00 | 4.15 | 0.00 | 1.22¹ |
| (n per 100 patient years) | | | | |

¹P < 0.001 Eculizumab Treatment vs. Pre-Treatment

The apparent heterogeneity in the different pre-study TE event rates may have been related in part to the different inclusion criteria of the three individual studies and/or the different sites involved in the individual studies. However, as opposed to earlier reports, current TE event ascertainment was systematic, prospective, and performed on a multicenter, international, and controlled basis in the C04-001, C04-002, and E05-001 studies. For these reasons, it is likely that the current pre-study TE event rates more likely represent the TE event rate in this PNH patient population prior to enrollment in the eculizumab PNH studies, although even these estimates may underestimate the true TE event rate as discussed below. Further, despite heterogeneity in the individual pre-study TE event rates, eculizumab treatment consistently resulted in a marked reduction in TE event rate in each individual study.

Tests for Robustness of Eculizumab Effect on Thrombosis

Because of the striking reduction in TE event rate observed above, five post-hoc analyses were performed to test the robustness of the observed effect. The major confounding issues that were identified were possible reduction in TE event rate reporting in the randomized clinical trial as compared to the medical history, reduction in TE event rates over time during the pre-eculizumab treatment period, quantitative imbalance between the pre-eculizumab treatment and treatment period quantity of patient years, impact of eculizumab treatment on patients with previous TE, reduction in TE event rates during the pre-eculizumab treatment period due to concomitant anticoagulant therapy.

Evaluation of Potential for Reduction in TE Event Rate Reporting in the Randomized Clinical Trial as Compared to the Medical History

In order to control for the potential confounding impact of an unexpected reduction in TE reporting during the randomized clinical trial as compared to pre-enrollment medical history, TE events were compared pre-enrollment and in placebo-treated C04-001 patients.

The TE event rate in patients treated with placebo was not reduced when compared to the rate in the same patients prior to placebo treatment. The TE event rate was 2.34 events per 100 patient years pre-placebo treatment and 4.38 events per 100 patient years in the same patients with placebo treatment. Thromboembolic event rates are shown in Table 4.

This analysis does not support the view that there was any intrinsic reduction in TE event rate reporting during the eculizumab clinical studies.

TABLE 4
Thromboembolic Events in the Same Patients Prior to Placebo/
Treatment and with Placebo Treatment in C04-001

| | C04-001 |
|---|---|
| Pre-Treatment | |
| Patients (n) | 44 |
| MAVE Events (n) | 11 |
| Patient Years (n) | 470.4 |
| MAVE Event Rate (n per 100 patient years) | 2.34 |
| Placebo Treatment | |
| Patients (n) | 44 |
| MAVE Events (n) | 1 |
| Patient Years (n) | 22.9 |
| MAVE Event Rate (n per 100 patient years) | 4.38 |

Twelve Months before Treatment vs. Eculizumab Treatment:

In order to evaluate for the potential of both (i) an unexpected reduction in the TE event rate immediately preceding trial entry, and also (ii) a quantitative imbalance between the quantity of patient years associated with the pre-eculizumab treatment and eculizumab treatment periods, a single analysis was performed; pre-eculizumab treatment TE event rates were truncated and only examined during the 12 months immediately preceding eculizumab treatment and compared to the available eculizumab treatment period. This single analysis served to both remove more distant years from the analysis and therefore focus more so on the most recent medical condition of the patients and to also equalize the quantity of patient years considered in the analysis prior to treatment with the quantity of patient years currently available with eculizumab treatment.

Compared to the TE event rate during only the 12 month period immediately preceding commencement of eculizumab treatment, eculizumab treatment resulted in a reduction in TE event rate in the same patients in each of the individual clinical studies and a significant reduction in the TE event rate overall. The TE event rate was reduced from 17.21 events per 100 patient years pre-eculizumab treatment to 1.22 events per 100 patient years in the same patients with eculizumab treatment (P=0.013). This represented a relative reduction of 93%, and an absolute reduction of 15.99 TE events per 100 patient years. Thromboembolic event rates are shown in Table 5.

It is noteworthy that the TE event rate in the 12 month period immediately preceding eculizumab treatment is markedly increased at 17.21 TE events per 100 patient years, as compared to the aggregate event rate of 7.49 TE events per 100 patient years for the entire period of time extending from the earlier of first TE/PNH diagnosis to enrollment into one of the eculizumab PNH trials. Thus, the data demonstrate that the TE event rates during the 12 month period immediately preceding eculizumab treatment were not reduced as compared to the overall pre-eculizumab treatment event rate. This crescendo TE event rate immediately preceding trial enrollment may be indicative of a substantial survivor bias in the pre-eculizumab treatment dataset. Additionally, this crescendo pattern of the TE event rate in the period immediately preceding commencement of eculizumab treatment was followed by a comparative arrest of the TE event rate with eculizumab treatment.

Truncating the analysis to equalize the quantity of patient years in the two comparison groups, as shown in Table 5 below, did not mitigate the observed beneficial impact of eculizumab on the TE event rate. With an approximately equal quantity of patient years distributed before and during eculizumab treatment, the observed relative and absolute reductions in TE event rates with eculizumab treatment were, if anything, greater than those observed with the primary analysis.

TABLE 5
Thromboembolic Events in Patients during the 12 Months Prior to
Start of Eculizumab Treatment and During Eculizumab Treatment in
C04-001, C04-002, C02-001/E02-001/X03-001 and E05-001

| | | | C02-001/ | E05-001 |
|---|---|---|---|---|
| | | C04- | E02-001/ | (All studies |
| | C04-001 | 002 | X03-001 | combined) |
| Pre-Treatment | | | | |
| Patients (n) | 43 | 97 | 11 | 195 |
| MAVE Events (n) | 6 | 23 | 3 | 33 |
| Patient Years (n) | 42.9 | 93.8 | 11.0 | 191.8 |
| MAVE Event Rate | 13.98 | 24.51 | 27.27 | 17.21 |
| (n per 100 patient years) | | | | |
| SOLIRIS ™ Treatment | | | | |
| Patients (n) | 43 | 97 | 11 | 195 |
| MAVE Events (n) | 0 | 2 | 0 | 2 |
| Patient Years (n) | 21.8 | 48.2 | 35.8 | 164.1 |
| MAVE Event Rate | 0.00 | 4.15 | 0.00 | 1.22¹ |
| (n per 100 patient years) | | | | |

¹P = 0.013 Eculizumab vs. Pre-Treatment

Evaluation of Impact of Eculizumab Treatment on Patients with Previous TE

In order to control for and identify the impact of previous thrombosis on the analysis, the effect of eculizumab on the TE event rate in patients with previous TE was examined. Patients who did not have a TE event pre-eculizumab treatment were excluded from the analysis.

Compared to the rate of thromboembolic events in patients with previous TE before eculizumab treatment, eculizumab treatment resulted in a reduction in TE event rate in the same patients in each of the individual clinical studies and a significant reduction in TE event rate overall. The TE event rate was reduced from 21.95 TE events per 100 patient years pre-eculizumab treatment to 3.42 TE events per 100 patient years in the same patients with eculizumab treatment (P<0.001). This represented a reduction of 84%, and an absolute reduction of 18.53 TE events per 100 patient years. Thromboembolic event rates are shown in Table 6.

Thus, in patients with the highest TE event rate pre-eculizumab treatment, eculizumab treatment caused a commensurate and highly significant reduction in TE event rates.

TABLE 6
Thromboembolic Events in Patients with Previous Thrombotic Events
Prior to Start of Eculizumab Treatment and During Eculizumab Treatment
in C04-001, C04-002, C02-001/E02-001/X03-001 and E05-001

| | | | C02-001/ | E05-001 |
|---|---|---|---|---|
| | | C04- | E02-001/ | (All studies |
| | C04-001 | 002 | X03-001 | combined) |
| Pre-Treatment | | | | |
| Patients (n) | 9 | 42 | 3 | 63 |
| MAVE Events (n) | 16 | 93 | 5 | 126 |
| Patient Years (n) | 78.7 | 329.2 | 17.6 | 574.2 |
| MAVE Event Rate | 20.34 | 28.25 | 28.43 | 21.95 |
| (n per 100 patient years) | | | | |
| Eculizumab Treatment | | | | |
| Patients (n) | 9 | 42 | 3 | 63 |
| MAVE Events (n) | 0 | 2 | 0 | 2 |
| Patient Years (n) | 4.6 | 20.7 | 9.1 | 58.5 |
| MAVE Event Rate | 0.00 | 9.68 | 0.00 | 3.42¹ |
| (n per 100 patient years) | | | | |

¹P < 0.001 Eculizumab vs. Pre-Treatment

Evaluation of Impact of Eculizumab Treatment on Patients Treated Concomitantly with Anticoagulant Therapy

In order to evaluate the potential impact of other anti-thrombotic therapies (which can comprise both anticoagulant and anti-platelet therapy) to reduce TE event rates over time prior to eculizumab treatment, the potentially confounding effect of anticoagulant therapy was controlled by specifically examining the effect of eculizumab treatment on the TE event rate in patients with previous anticoagulation therapy. TE event rates in patients who were never anticoagulated were also examined; in these analyses, TE events prior to initiation of anticoagulant therapy were excluded.

Compared to the TE event rate in patients treated with anticoagulant therapy before commencement of eculizumab treatment, eculizumab treatment resulted in a reduction in the TE event rate in the same patients in each of the individual clinical studies and a significant reduction in the TE event rate overall. The TE event rate was reduced from 14.00 TE events per 100 patient years with anticoagulant therapy but prior to commencement of eculizumab treatment to 0.00 TE events per 100 patient years with eculizumab treatment in the same patients (P<0.001). This represented a relative reduction of 100%, and an absolute reduction of 14.00 TE events per 100 patient years. Thromboembolic event rates are shown in Table 7.

Compared to the negligible rate of thromboembolic events in patients without anticoagulant therapy before eculizumab treatment, eculizumab treatment resulted in no meaningful change in the thrombotic event rate. The TE event rate was 1.31 TE events per 100 patient years pre-eculizumab treatment and 2.90 TE events per 100 patient years in the same patients with eculizumab treatment (P=1.000). Thromboembolic event rates are shown in Table 8.

TABLE 7
Thromboembolic Events in Patients with Previous Anticoagulant
Treatment Prior to Start of Eculizumab Treatment and During
Eculizumab Treatment in C04-001, C04-002, C02-001/E02-001/X03-001
and E05-001

| | | | C02-001/ | E05-001 |
|---|---|---|---|---|
| | | C04- | E02-001/ | (All studies |
| | C04-001 | 002 | X03-001 | combined) |
| Pre-Treatment | | | | |
| Patients (n) | 23 | 51 | 9 | 103 |
| MAVE Events (n) | 11 | 35 | 4 | 54 |
| Patient Years (n) | 72.7 | 168.6 | 45.9 | 385.7 |
| MAVE Event Rate | 15.13 | 20.76 | 8.71 | 14.00 |
| (n per 100 patient years) | | | | |
| Eculizumab Treatment | | | | |
| Patients (n) | 23 | 51 | 9 | 103 |
| MAVE Events (n) | 0 | 0 | 0 | 0 |
| Patient Years (n) | 11.9 | 24.8 | 28.8 | 100.1 |
| MAVE Event Rate | 0.00 | 0.00 | 0.00 | 0.00¹ |
| (n per 100 patient years) | | | | |

¹P < 0.001 Eculizumab vs. Pre-Treatment

TABLE 8
Thromboembolic Events in Patients without Previous Anticoagulant
Treatment Prior to Start of Eculizumab Treatment and During
Eculizumab Treatment in C04-001, C04-002, C02-001/E02-001/X03-001
and E05-001

| | | | C02-001/ | E05-001 |
|---|---|---|---|---|
| | | C04- | E02-001/ | (All studies |
| | C04-001 | 002 | X03-001 | combined) |
| Pre-Treatment | | | | |
| Patients (n) | 20 | 46 | 2 | 92 |
| MAVE Events (n) | 0 | 7 | 0 | 10 |
| Patient Years (n) | 122.4 | 319.4 | 69.0 | 764.3 |
| MAVE Event Rate | 0.00 | 2.19 | 0.00 | 1.31 |
| (n per 100 patient years) | | | | |
| Eculizumab Treatment | | | | |
| Patients (n) | 20 | 46 | 2 | 92 |
| MAVE Events (n) | 0 | 2 | 0 | 2 |
| Patient Years (n) | 9.9 | 22.2 | 7.0 | 69.0 |
| MAVE Event Rate | 0.00 | 8.99 | 0.00 | 2.90¹ |
| (n per 100 patient years) | | | | |

¹P = 1.000 Eculizumab vs. Pre-Treatment

Eculizumab has been demonstrated to be safe and well tolerated for the treatment of PNH. In studies of patients diagnosed with PNH, there were no apparent significant safety concerns associated with eculizumab therapy. Adverse event frequency was similar in eculizumab and placebo-treated patients and the overall frequency of serious adverse events was less with eculizumab than with placebo. There was one reported infection with Neisseria species in a vaccinated PNH patient that was treated effectively and resolved without clinical sequelae. The overall frequency of infections was similar with eculizumab and placebo. Serious hemolysis following discontinuation of eculizumab in PNH patients was not observed and patients that discontinued eculizumab were effectively managed by standard of care. The incidence of bone marrow failure disorders was unchanged with eculizumab treatment. In addition, no dose-related toxicities were observed in these studies.

### Example 3

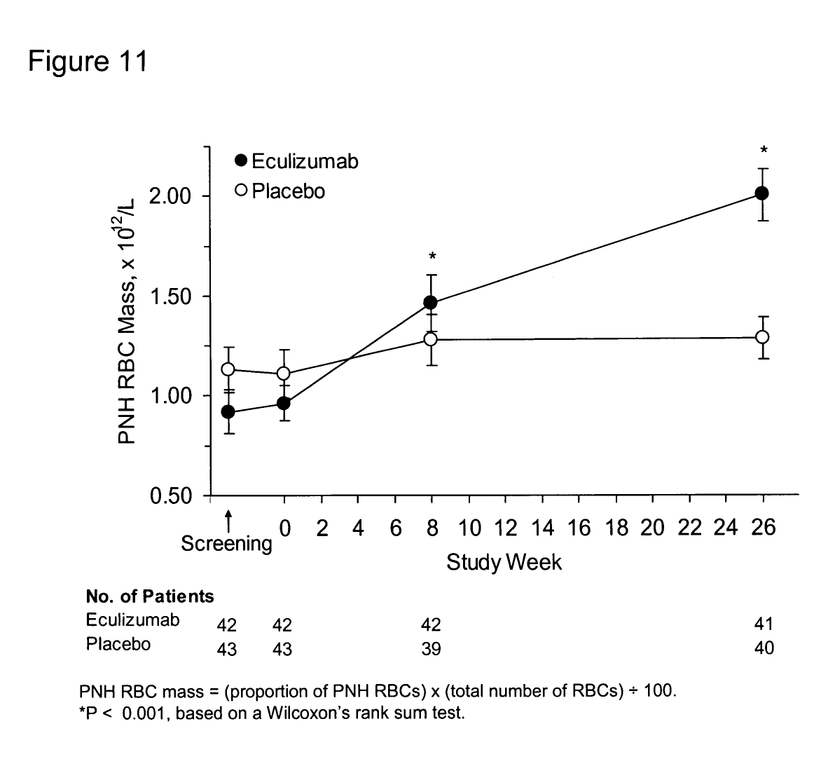

Eculizumab, a complement inhibitor, was shown to reduce intravascular hemolysis and transfusion requirements in patients with PNH. Eculizumab-treated patients, as compared to placebo, showed an 85.8% decrease in intravascular hemolysis (as measured by LDH area under a curve, p<0.001). This reduction in hemolysis with eculizumab resulted in a 2.5-fold increase in PNH RBC mass from a median of 0.81×10¹² cells/L at baseline to 2.05×10¹² cells/L at 26 weeks (p<0.001), while the PNH RBC mass in placebo-treated patients remained relatively unchanged (from a median of 1.09×10¹² cells/L to 1.16×10¹² cells/L) (FIG. 11). The increase in PNH RBC mass was associated with an overall increase in hemoglobin levels in eculizumab-treated patients relative to placebo (p<0.001, mixed model analysis). The number of PRBC units transfused decreased from a median of 10.0/patient with placebo to 0.0/patient with eculizumab (p<0.001), and 51.2% of eculizumab-treated patients became transfusion independent (versus 0.0% of placebo patients, p<0.001). Even patients who required some transfusions while on eculizumab showed a marked reduction in transfusion requirements from a median of 10.0 units per patient with placebo to 6.0 units/patient with eculizumab (p<0.001). The reduction in PRBC units transfused with eculizumab was observed regardless of transfusion requirements prior to treatment, with statistical significance reached in 3 of 3 pre-treatment transfusion strata (4 to 14 units/year; 15-25 units/year; and>25 units/year, p<0.001 for each stratum) (see Table 9). Significant reductions were observed in intravascular hemolysis (LDH) in eculizumab-treated patients that achieved transfusion independence (p<0.001) as well as those that did not (p<0.001) (see Table 10). Taken together, these data demonstrate that effective control of intravascular hemolysis in PNH with eculizumab results in a substantial improvement in anemia, as evidenced by an increase in endogenous RBC mass, an improvement in hemoglobin levels, and a reduction in transfusion requirements. Substantial and significant reductions in intravascular hemolysis and improvements in anemia with eculizumab are demonstrated regardless of historical transfusion requirements or whether patients achieve transfusion independence during treatment. See Hillmen et al., N. Engl. J. Med. 355:1233-1243 (2006).

TABLE 9
Transfusion Requirement during Treatment by Pretreatment
Transfusion Strata

| | | Median Packed Red Cells Transfused | | |
|---|---|---|---|---|
| Transfusion | | (units/patient) | | |
| Stratum (Units) | No. Patients | Placebo | Eculizumab | P value* |
| Overall | 87 | 10.0 | 0.0 | <0.001 |
| 4-14 | 30 | 6.0 | 0.0 | <0.001 |
| 15-25 | 35 | 10.0 | 2.0 | <0.001 |
| >25 | 22 | 18.0 | 3.0 | <0.001 |

TABLE 10
Hemolysis (LDH AUC) during Treatment by Pretreatment
Transfusion Strata

| | | Median Lactate Dehydrogenase Area | | |
|---|---|---|---|---|
| Transfusion | | under the Curve (Units/L × Day) | | |
| Stratum (Units) | No. Patients | Placebo | Eculizumab | P value* |
| Overall | 87 | 411,822 | 58,587 | <0.001 |
| 4-14 | 30 | 398,573 | 53,610 | <0.001 |
| 15-25 | 35 | 420,338 | 56,127 | <0.001 |
| >25 | 22 | 441,880 | 67,181 | <0.001 |

Randomization strata were based on transfusion data over a period of 12-months prior to screening.
*P value was calculated using Wilcoxon's rank sum test.

### Example 4

A 48-year old transfusion-dependent male was diagnosed with aplastic anemia in May 1988 and with PNH in September 1993. He has been transfusion dependent due to PNH starting in September 1993, requiring transfusions of packed red blood cells (PRBCs) every 4 to 6 weeks. He received eculizumab infusions starting May 22, 2002 and is currently dosed at 900 mg every other week. On Nov. 6, 2002, after 6 months of receiving eculizumab, rHuEpo (NeoRecormon®) therapy was initiated at the following doses: 450 IU/kg/week in 3 divided doses during the first 2 months; 900 IU/kg/week in 3 divided doses during the next 15 months; and 750 IU/kg/week in 3 divided doses until May 3, 2006. At that time he was switched to Aranesp® at a dose of 300 mcg every 2 weeks. The dose was increased to 500 mcg every 2 weeks on 28th Jun. 2006.

Intravascular hemolysis was assessed by measuring levels of the enzyme lactate dehydrogenase (LDH). Levels of erythropoiesis were determined by measuring reticulocyte counts. PNH RBC mass was calculated by multiplying the absolute number of RBCs by the proportion of PNH type III RBCs as assessed by flow cytometry. Hemoglobin levels and PRBC transfusion requirements were also monitored. All assessments have been collected to the present date and results are reported through August 2006.

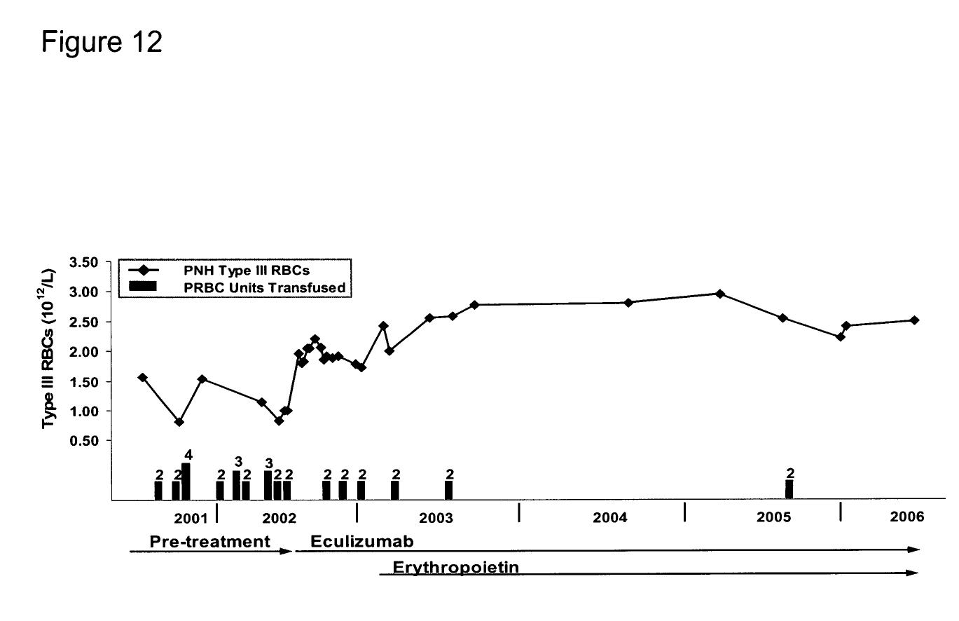

During the year prior to eculizumab therapy, the mean LDH level was 2,075 IU/L (more than 4 times that of the upper limit of the normal range), the mean hemoglobin level was 10.5 g/dL, and the mean reticulocyte count was 77.5×10⁹/L (Table II). The absolute number of PNH type III RBCs was 1.1×10¹²/L, and the proportion of these cells constituted less than 50% of the total RBC mass. The patient required 1.8 units of PRBCs per month during the pre-treatment period (Table 11), receiving a total of 9 transfusions and 22 units (FIG. 12).

TABLE 11
Hematological Parameters Before and After Eculizumab and rHuEpo
Therapies.

| | Mean ± SD | | |
|---|---|---|---|
| | | Eculizumab | Eculizumab + |
| | Pre-treatment | alone | RHuEPO |
| Parameter | (1 year) | (0.5 year) | (3.7 years) |
| LDH, IU/L (normal | 2075 ± 1590 | 456 ± 76 | 679 ± 146 |
| range 150-480) | | | |
| Hemoglobin, g/dL | 10.5 ± 1.5 | 10.2 ± 0.9 | 11.4 ± 1.1 |
| (normal range 13.5-18.0) | | | |
| Reticulocytes, ×10⁹/ | 77.5 ± 10.6 | 96.4 ± 29.5 | 205.3 ± 43.6 |
| L (normal range 20-80) | | | |
| PNH type III | 1.1 ± 0.3 | 1.9 ± 0.1 | 2.5 ± 0.3 |
| RBCs, ×10¹²/L* | | | |
| Units transfused per | 1.8 | 1.0 | 0.1 |
| month | | | |

*Calculated as (proportion of PNH type III RBCs) × (total number of RBCs) ÷ 100

After starting eculizumab treatment, hemolysis was rapidly and consistently reduced as indicated by a 78% decrease in the mean LDH level (Table 11). A concomitant increase (73%) in the PNH type III RBC mass was also demonstrated, supporting enhanced survival of these cells. Further, the average number of transfusions required each month was reduced by 44%. RBC hemoglobin was stable even though transfusion requirement decreased, indicating a net increase in endogenous hemoglobin levels (Table 11).

After 6 months of eculizumab treatment, the patient received concomitant rHuEpo therapy resulting in a mean reticulocyte count increase of 113% (Table 11). This increase in erythropoiesis was associated with an additional 32% increase in the PNH type III RBC mass over that achieved with eculizumab treatment alone. In addition, RBC hemoglobin levels showed an increase from 10.2 g/dL to 11.4 g/dL during the same period. This improvement in anemia resulted in a further decrease in transfusion requirements, eventually leading to transfusion-independence for more than two years (FIG. 12). One transfusion was given after the two-years of transfusion independence and this coincided with a transient decrease in erythropoiesis, as evidenced by a drop in the reticulocyte count (data not shown). There was no evidence-of an increase in intravascular hemolysis and LDH levels have remained within the normal range or just above the upper limit of the normal range during the entire treatment period. This patient continues to receive eculizumab and rHuEpo and has received only 1 transfusion in more than 3 years.

### Example 5

Pulmonary hypertension (PHT) is an emerging common complication of hereditary hemolytic anemias. It has been mechanistically and epidemiologically linked to intravascular hemolysis and decreased nitric oxide (NO) bioavailability. While this complication has been described in approximately 30% of adult patients with sickle cell disease and thalassemia, the prevalence of PHT in patients with paroxysmal nocturnal hemoglobinuria (PNH), an acquired disease with the highest levels of intravascular hemolysis observed, has never been determined. PNH patients frequently have symptoms consistent with both hemolysis and PHT including severe fatigue and dyspnea on exertion. Therefore, we examined for the presence of PHT in PNH and explored potential mechanisms associated with its development by measuring the ability of plasma to instantaneously consume NO using ozone-based chemiluminescence.

Doppler echocardiography was performed in 28 hemolytic PNH patients to estimate pulmonary artery systolic pressures. Systolic PHT was defined by a tricuspid regurgitant jet velocity (TRV)≧2.5m/s at rest. Fourteen (50%) patients had elevated pulmonary artery systolic pressures. Twelve (43%) had mild to moderate PHT (mean TRV 2.6m/s±0.01) while two (7%) had moderate to severe pressures (mean TRV 3.7m/s±0.02). Plasma from PNH patients (n=32) consumed 34.6±8.3μM NO while normal subjects (n=9) consumed 2.2±0.6μM NO (p=0.0001). LDH levels correlated with NO consumption (r=0.6342, p<0.0002). In a separate cohort of 7 patients treated with eculizumab for a median of 3 years to reduce hemolysis, the ability to consume NO appeared lower (13.2±4.8 μM NO).

### Example 6

PNH patients suffer from diverse and serious hemolysis-induced morbidities leading to a poor quality of life (QoL). Fatigue in PNH patients may be disabling and levels are similar to anemic cancer patients. Fatigue is multifactoral, related to both the underlying anemia and hemolysis. Patients suffer from reduced global health status, patient functioning, pain and dyspnea. Treatment with the complement inhibitor eculizumab reduces intravascular hemolysis and improves anemia. The impact of eculizumab treatment on levels of fatigue and other patient reported outcomes was prospectively examined in a double-blind placebo-controlled study (TRIUMPH) using two distinct instruments, the FACIT-Fatigue and the EORTC QLQ-C30. Improvements in QoL were quantified using standardized effect sizes (SES), a measure of the magnitude of the clinical benefit in various instruments. Eculizumab treatment, as compared to placebo, was associated with a very large and significant improvement in fatigue as measured by the FACIT-fatigue scale (SES=1.13, P<0.001) as well as the EORTC-QLQ-C30 fatigue subscale ((SES=1.12, P<0.001). Similarly, the percentage of patients achieving a pre-specified minimally important difference (MID) was 53.7% versus 20.5% of eculizumab- and placebo-treated patients, respectively (P=0.003) using the FACIT-Fatigue; 67.6% versus 24.4%, respectively (P<0.001) with the EORTC QLQ-C30. Treatment independent univariate analyses showed that reduction in intravascular hemolysis (decreased LDH levels) and improvement in anemia (increased hemoglobin levels) were both significantly associated with an improvement in fatigue. Further multivariate analyses indicated that reduction in hemolysis was more predictive than improvement in anemia of an improvement in fatigue. Eculizumab treatment was also associated with significant improvements with moderate to large SES in the following EORTC-QLQ-C30 subscales: global health status (0.87, P<0.001); role functioning (0.93, P<0.001); social functioning (0.57, P=0.003); cognitive functioning (0.78, P=0.002); physical functioning (1.01, P<0.001); emotional functioning (0.51, P=0.008); pain (0.65, P=0.002); dyspnea (0.69, P<0.001); and appetite loss (0.50, P<0.001). These data demonstrate that resolution of intravascular hemolysis with eculizumab treatment results in large and clinically meaningful improvements in patient reported outcomes including fatigue, global health status, patient functioning, and disease-related symptoms in PNH.

### Example 7

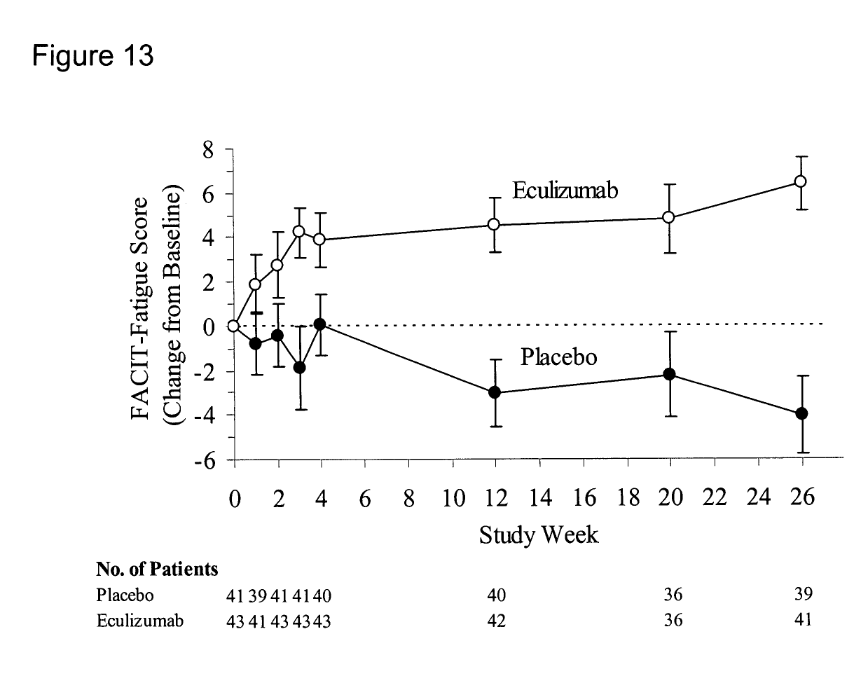

In paroxysmal nocturnal hemoglobinuria (PNH), lack of the GPI-anchored terminal complement inhibitor CD59 from blood cells renders erythrocytes susceptible to chronic hemolysis resulting in anemia, fatigue, thrombosis, poor quality of the life (QoL), and a dependency on transfusions. Eculizumab, a complement inhibitor, reduced intravascular hemolysis and transfusion requirements in transfusion dependent patients with normal or near-normal platelet counts in a randomized placebo-controlled trial (TRIUMPH). SHEPHERD, an open-label, non-placebo controlled 52-week phase III clinical study, is underway to evaluate the safety and efficacy of eculizumab in a broader PNH population including patients with significant thrombocytopenia and/or lower transfusion requirements. Eculizumab was dosed as follows: 600 mg IV every 7 days×4; 900 mg 7 days later; and then 900 mg every 14±2 days. Eculizumab was administered to 97 patients at 33 international sites. In a pre-specified 6-month interim analysis, the most frequent adverse events were headache (50%), nasopharyngitis (23%), and nausea (16%); most were mild to moderate in severity. No infections or serious adverse events were reported as “probably” or “definitely” related to drug. Intravascular hemolysis, the central clinical manifestation in PNH and the primary surrogate efficacy endpoint of the trial, was significantly reduced in eculizumab patients as assessed by change in lactate dehydrogenase (LDH) area under the curve (p<0.001). LDH levels decreased from a median of 2,051 U/L at baseline to 270 U/L at 26 weeks (p<0.001; normal range 103-223 U/L). Control of intravascular hemolysis resulted in an improvement in anemia as transfusion requirements decreased from a median of 4.0 PRBC units/patient pre-treatment to 0.0 during treatment (p<0.001), approximately 50% of the patients were rendered transfusion independent (P<0.001), and hemoglobin levels increased (p<0.001). Fatigue, as measured by both the FACIT-Fatigue and EORTC QLQ-C30 instruments, was significantly improved with eculizumab treatment as compared to baseline (p<0.001 for each) (FIG. 13 and Table 12). Other EORTC-QLQ-C30 patient reported outcomes demonstrating improvement included global health status (p<0.001), all 5 patient functioning subscales (p<0.001) and 7 of 9 symptom/single item subscales (p<0.03). These results demonstrate that the beneficial effects of eculizumab in PNH are applicable to a much broader patient population than previously studied and further underscore that eculizumab treatment markedly reduces intravascular hemolysis, thereby providing clinical benefit to treated patients.

TABLE 12
Fatigue and other outcomes, as measured by both the FACIT-Fatigue
and EORTC QLQ-C30 instruments.

| | | | Change from |
|---|---|---|---|
| | | | Baseline |
| | MID (%)* | SES† | P-Value‡ |
| FACIT-Fatigue | 74.5 | 1.01 | <0.001 |

EORTC QLQ-C30

| | | | |
|---|---|---|---|
| Fatigue | 80.9 | 1.08 | <0.001 |
| Global Health Status | 59.6 | 0.73 | <0.001 |

Functioning scales

| | | | |
|---|---|---|---|
| Physical | 50.0 | 0.86 | <0.001 |
| Role | 55.3 | 0.70 | <0.001 |
| Cognitive | 39.4 | 0.40 | <0.001 |
| Social | 54.3 | 0.61 | <0.001 |
| Emotional | 44.7 | 0.58 | <0.001 |
| Dyspnea | 55.3 | 0.75 | <0.001 |
| Pain | 30.9 | 0.30 | 0.004 |
| Appetite loss | 20.2 | 0.31 | <0.001 |
| Insomnia | 35.1 | 0.48 | <0.001 |
| Financial difficulties | 15.1 | 0.08 | 0.804 |
| Constipation | 10.8 | 0.08 | 0.758 |
| Nausea/vomiting | 18.1 | 0.05 | 0.034 |
| Diarrhea | 17.0 | 0.27 | <0.001 |

### Example 8

Paroxysmal nocturnal hemoglobinuria (PNH) is characterized by clonal expansion of PNH red cells that are highly sensitive to lysis by terminal complement. The primary lesion in PNH is bone marrow failure in the form of immune-mediated aplastic anemia and peripheral blood cytopenias of varying severity. In Example 1, the successful control of hemolysis and transfusion in 11 patients with the complement inhibitor eculizumab is described. Ten of these 11 patients remained on eculizumab therapy after approximately 3 years with maintained reductions in intravascular hemolysis and transfusion. The effectiveness of eculizumab therapy in these patients is through the protection of the PNH red cell from complement-mediated lysis and the expansion of this cell population. Flow cytometry studies have shown that the percentage of PNH red cells increased significantly from a mean of 36.7% before treatment to 58.4% at week 64 of therapy. Importantly, granulocyte, monocyte and platelet PNH clone sizes were>90% before treatment and remained stable for all patients throughout the trial suggesting that the majority of hematopoiesis is derived from PNH stem cells. It is hypothesized that the PNH red cell clone should approach the clone size of other myeloid hematopoietic cells in a given patient when hemolysis is prevented by eculizumab therapy as this more accurately depicts PNH stem cell activity.

In all patients hemolysis was substantially reduced by 21 days. In 9 of 1 patients, there was a rapid rise in PNH red cell count with the mean absolute number of PNH red cells increasing from 1.37×10¹²/L before treatment to 1.50×10¹²/L at 2 weeks (P=0.21), 1.74×10¹²/L at 4 weeks (P=0.002), and 2.11×10¹²/L at 12 weeks (P=0.001) of eculizumab treatment. The maximum theoretical red cell response was achieved in a mean of 178 days (range 49-419 days). The mean absolute number of PNH red cells increased to 2.37×10¹²/L at maximum response (P=0.001), an increase of 73% (range 36% -207%). All patients achieved a maximum response prior to 18 months of treatment and clone size was subsequently stable. In 2 patients, despite the effectiveness of eculizumab in resolving hemolysis, there was no change in absolute numbers of PNH red cells pre and post-treatment. This is likely due to a combination of a lower degree of hemolysis and more profound bone marrow insufficiency in these patients. The determination of absolute PNH red cell counts during the first 12 months of eculizumab therapy may identify which patients will become transfusion independent and which patients may benefit from additional growth factor support to boost erythropoiesis. Furthermore, long-term eculizumab therapy appeared to be associated with a stable PNH red cell clone size in this initial clinical study.

### Example 9

Surprisingly, in the C04-002 study, eculizumab treatment, as compared to baseline, was associated with an apparent increase in parameters of platelet activation (mixed model analysis, overall). Statistically significant increases were observed in monocyte-platelet aggregates (mean increase of 7.9%, P=0.002), neutrophil-platelet aggregates (mean increase of 5.3%, P<0.001) and the percentage of P-Selectin positive platelets (mean increase of 3.7%, P<0.001). Similarly, in eculizumab-treated patients in the C04-001 study increases were observed in monocyte-platelet aggregates (mean increase of 15.0%, P=0.056), neutrophil-platelet aggregates (mean increase of 11.2%, P=0.777) and the percentage of P-Selectin positive platelets (mean increase of 5.1%, P=0.044).

In the combined C04-001 and C04-002 studies, significant increases were also observed in monocyte-platelet aggregates (mean increase of 10.1%, P<0.00 1), neutrophil-platelet aggregates (mean increase of 7.0%, P<0.001) and the percentage of P-Selectin positive platelets (mean increase of 4.1%, P<0.001). In this placebo-controlled C04-001 study, the eculizumab cohort as well as the placebo cohort showed similar increases in parameters of platelet activation from baseline (Table 13A-F). In these placebo-treated patients, increases from baseline were observed in monocyte-platelet aggregates (mean increase of 2.2%, P=0.771), neutrophil-platelet aggregates (mean increase of 6.9%, P=0.135) and the percentage of P-Selectin positive platelets (mean increase of 5.9%, P−0.001) (Table 14A-C).

TABLE 13A
C04-002; C04-001 and C04-002 Compared and Combined: Mixed model
analysis of change in Platelet Activation Markers; Change from Baseline
MONOCYTE PLATELET AGGREGATION

| Study | | Study Week | | Statistic | DF | P Value(a) |
|---|---|---|---|---|---|---|
| C04-001 (N = 43) | Mean Baseline | | 44.282 | | | |
| | Value | | | | | |
| | Least Square | 1 | 9.294 | 0.98 | 34 | 0.333249423 |
| | Means | 2 | 14.009 | 1.48 | 34 | 0.148210796 |
| | | 4 | 27.935 | 2.95 | 34 | 0.005713194 |
| | | 14 | 16.012 | 1.64 | 34 | 0.110194214 |
| | | 26 | 10.827 | 1.11 | 34 | 0.275177698 |
| | Overall(b) | Overall | 15.005 | 1.98 | 34 | 0.055927296 |
| C-04-002 (N = 97) | Mean Baseline | | 26.310 | | | |
| | Value | | | | | |
| | Least Square | 1 | 2.967 | 0.65 | 95 | 0.518317428 |
| | Means | 2 | 2.691 | 0.62 | 95 | 0.534704269 |
| | | 4 | 9.596 | 2.22 | 95 | 0.028644828 |
| | | 14 | 11.193 | 2.54 | 95 | 0.012540534 |
| | | 26 | 11.838 | 2.69 | 95 | 0.008417504 |
| | Overall(b) | Overall | 7.940 | 3.17 | 95 | 0.002037650 |

NOTE:
(a)P value was based on T test.
(b)Overall was based on the average of least square means from Week 1 to Week 26.

TABLE 13B
C04-002; C04-001 and C04-002 Compared and Combined: Mixed model
analysis of change in Platelet Activation Markers; Change from Baseline
MONOCYTE PLATELET AGGREGATION

| Study | | Study Week | | Statistic | DF | P Value(a) |
|---|---|---|---|---|---|---|
| Combined (N = 140) | Mean Baseline | | 31.302 | | | |
| | Value | | | | | |
| | Least Square | 1 | 4.971 | 1.15 | 133 | 0.250281818 |
| | Means | 2 | 5.794 | 1.39 | 133 | 0.166169537 |
| | | 4 | 14.650 | 3.52 | 133 | 0.000591120 |
| | | 14 | 12.842 | 3.02 | 133 | 0.003059012 |
| | | 26 | 11.824 | 2.78 | 133 | 0.006256488 |
| | Overall(b) | Overall | 10.092 | 3.47 | 133 | 0.000691639 |
| Placebo (N = 44) | Mean Baseline | | 39.444 | | | |
| | Value | | | | | |
| | Least Square | 1 | 4.768 | 0.55 | 44 | 0.585815062 |
| | Means | 2 | −3.814 | −0.45 | 44 | 0.652851604 |
| | | 4 | 5.484 | 0.63 | 44 | 0.531000233 |
| | | 14 | −4.964 | −0.55 | 44 | 0.583493606 |
| | | 26 | 13.523 | 1.61 | 44 | 0.115481853 |
| | Overall(b) | Overall | 2.181 | 0.37 | 44 | 0.711338659 |

NOTE:
(a)P value was based on T test.
(b)Overall was based on the average of least square means from Week 1 to Week 26.

TABLE 13C
C04-002; C04-001 and C04-002 Compared and Combined: Mixed model analysis of
change in Platelet Activation Markers; Change from Baseline
NEUTROPHIL-PLATELET AGGREGATION

| Study | | Study Week | | Statistic | DF | P Value(a) |
|---|---|---|---|---|---|---|
| C04-001 (N = 43) | Mean Baseline | | 23.555 | | | |
| | Value | | | | | |
| | Least Square | 1 | 6.057 | 0.75 | 34 | 0.457636650 |
| | Means | 2 | 6.877 | 0.85 | 34 | 0.399618945 |
| | | 4 | 21.467 | 2.66 | 34 | 0.011756090 |
| | | 14 | 16.246 | 1.95 | 34 | 0.059947232 |
| | | 26 | 6.857 | 0.82 | 34 | 0.417142286 |
| | Overall(b) | Overall | 11.222 | 1.83 | 34 | 0.076671903 |
| C-04-002 (N = 97) | Mean Baseline | | 10.586 | | | |
| | Value | | | | | |
| | Least Square | 1 | 3.558 | 1.27 | 94 | 0.208460346 |
| | Means | 2 | 0.353 | 0.13 | 94 | 0.894343104 |
| | | 4 | 4.011 | 1.51 | 94 | 0.133423112 |
| | | 14 | 9.505 | 3.52 | 94 | 0.000663614 |
| | | 26 | 8.476 | 3.14 | 94 | 0.002261817 |
| | Overall(b) | Overall | 5.275 | 3.51 | 94 | 0.000679427 |

NOTE:
(a)P value was based on T test.
(b)Overall was based on the average of least square means from Week 1 to Week 26.

TABLE 13D
C04-002; C04-001 and C04-002 Compared and Combined: Mixed model
analysis of change in Platelet Activation Markers; Change from Baseline
NEUTROPHIL-PLATELET AGGREGATION

| Study | | Study Week | | Statistic | DF | P Value(a) |
|---|---|---|---|---|---|---|
| Combined (N = 140) | Mean Baseline | | 14.188 | | | |
| | Value | | | | | |
| | Least Square | 1 | 4.232 | 1.38 | 133 | 0.170014676 |
| | Means | 2 | 2.156 | 0.73 | 133 | 0.467887940 |
| | | 4 | 8.851 | 2.99 | 133 | 0.003344370 |
| | | 14 | 11.514 | 3.80 | 133 | 0.000220613 |
| | | 26 | 8.246 | 2.72 | 133 | 0.007382065 |
| | Overall(b) | Overall | 6.999 | 3.44 | 133 | 0.000781995 |
| Placebo (N = 44) | Mean Baseline | | 16.172 | | | |
| | Value | | | | | |
| | Least Square | 1 | 5.275 | 0.77 | 44 | 0.448005308 |
| | Means | 2 | −0.160 | −0.02 | 44 | 0.980942735 |
| | | 4 | 9.719 | 1.41 | 44 | 0.165084720 |
| | | 14 | 4.415 | 0.62 | 44 | 0.539239191 |
| | | 26 | 17.220 | 2.58 | 44 | 0.013226012 |
| | Overall(b) | Overall | 6.919 | 1.52 | 44 | 0.135363716 |

NOTE:
(a)P value was based on T test.
(b)Overall was based on the average of least square means from Week 1 to Week 26.

TABLE 13E
C04-002; C04-001 and C04-002 Compared and Combined: Mixed model
analysis of change in Platelet Activation Markers; Change from Baseline
P-SELECTIN EXPRESSION

| Study | | Study Week | | Statistic | DF | P Value(a) |
|---|---|---|---|---|---|---|
| C04-001 (N = 43) | Mean Baseline | | 7.941 | | | |
| | Value | | | | | |
| | Least Square | 1 | 2.157 | 0.50 | 34 | 0.621911155 |
| | Means | 2 | 5.479 | 1.26 | 34 | 0.214825093 |
| | | 4 | 7.478 | 1.73 | 34 | 0.093582884 |
| | | 14 | 6.591 | 1.45 | 34 | 0.157269235 |
| | | 26 | 6.011 | 1.32 | 34 | 0.195994953 |
| | Overall(b) | Overall | 5.149 | 2.09 | 34 | 0.044482961 |
| C-04-002 (N = 97) | Mean Baseline | | 7.517 | | | |
| | Value | | | | | |
| | Least Square | 1 | 4.462 | 2.12 | 95 | 0.036933290 |
| | Means | 2 | 4.009 | 2.02 | 95 | 0.046211034 |
| | | 4 | 3.817 | 1.92 | 95 | 0.057451135 |
| | | 14 | 2.535 | 1.25 | 95 | 0.213238625 |
| | | 26 | 3.035 | 1.50 | 95 | 0.137140051 |
| | Overall(b) | Overall | 3.671 | 3.45 | 95 | 0.000837199 |

NOTE:
(a)P value was based on T test.
(b)Overall was based on the average of least square means from Week 1 to Week 26.

TABLE 13F
C04-002; C04-001 and C04-002 Compared and Combined: Mixed model
analysis of change in Platelet Activation Markers; Change from Baseline
P-SELECTIN EXPRESSION

| Study | | Study Week | | Statistic | DF | P Value(a) |
|---|---|---|---|---|---|---|
| Combined (N = 140) | Mean Baseline | | 7.635 | | | |
| | Value | | | | | |
| | Least Square | 1 | 3.763 | 1.92 | 133 | 0.056451906 |
| | Means | 2 | 4.333 | 2.31 | 133 | 0.022390009 |
| | | 4 | 4.750 | 2.53 | 133 | 0.012475659 |
| | | 14 | 3.665 | 1.90 | 133 | 0.059358752 |
| | | 26 | 3.788 | 1.96 | 133 | 0.051519764 |
| | Overall(b) | Overall | 4.106 | 3.86 | 133 | 0.000178203 |
| Placebo (N = 44) | Mean Baseline | | 6.585 | | | |
| | Value | | | | | |
| | Least Square | 1 | 8.053 | 2.14 | 43 | 0.038297488 |
| | Means | 2 | 5.063 | 1.40 | 43 | 0.168857949 |
| | | 4 | 6.484 | 1.72 | 43 | 0.092313078 |
| | | 14 | 6.383 | 1.62 | 43 | 0.112358382 |
| | | 26 | 3.403 | 0.94 | 43 | 0.352152288 |
| | Overall(b) | Overall | 5.851 | 3.49 | 43 | 0.001115025 |

NOTE:
(a)P value was based on T test.
(b)Overall was based on the average of least square means from Week 1 to Week 26.

TABLE 14A
C04-001: Mixed Model Analyses of Platelet Activation Markers;
Change from Baseline
MONOCYTE-PLATELET AGGREGATION
Population: ITT

| | Degree of Freedom | | | |
|---|---|---|---|---|
| Effect | Numerator | Denominator | F Statistic | P Value |
| Baseline Platelet | 1 | 104 | 11.26 | 0.001105679 |
| Assay | | | | |
| Week | 5 | 104 | 1.33 | 0.255322788 |
| Treatment | 1 | 104 | 3.01 | 0.085849037 |

NOTE: Analysis based on North American ITT patients only as described in the protocol.

TABLE 14B
C04-001: Mixed Model Analyses of Platelet Activation Markers;
Change from Baseline
NEUTROPHIL-PLATELET AGGREGATION
Population: ITT

| | Degree of Freedom | | | |
|---|---|---|---|---|
| Effect | Numerator | Denominator | F Statistic | P Value |
| Baseline Platelet | 1 | 104 | 4.28 | 0.040938932 |
| Assay | | | | |
| Week | 5 | 104 | 2.20 | 0.060319683 |
| Treatment | 1 | 104 | 1.21 | 0.274747130 |

NOTE:
Analysis based on North American ITT patients only as described in the protocol.

TABLE 14C
C04-001: Mixed Model Analyses of Platelet Activation Markers;
Change from Baseline P-SELECTIN EXPRESSION
Population: ITT

| | Degree of Freedom | | | |
|---|---|---|---|---|
| Effect | Numerator | Denominator | F Statistic | P Value |
| Baseline Platelet | 1 | 103 | 9.01 | 0.003374798 |
| Assay | | | | |
| Week | 5 | 103 | 0.91 | 0.480577712 |
| Treatment | 1 | 103 | 0.26 | 0.613587524 |

NOTE:
Analysis based on North American ITT patients only as described in the protocol

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

What is claimed is:

1. A method of reducing the occurrence of thrombosis in a subject, said method comprising inhibiting complement in said subject.

2. The method of claim 1, wherein said method comprises administering a compound to said subject, wherein the compound is selected from the group consisting of: a) compounds which bind to one or more complement components, b) compounds which block the generation of one or more complement components, and c) compounds which block the activity of one or more complement components.

3. The method of claim 1, wherein said subject has a paroxysmal nocturnal hemoglobinuria (PNH) granulocyte clone greater than 0. 1% of the total granulocyte count.

4. The method of claim 1, wherein said subject has a PNH granulocyte clone greater than 1% of the total granulocyte count.

5. The method of claim 1, wherein said subject has a PNH granulocyte clone greater than 10% of the total granulocyte count.

6. The method of claim 1, wherein said subject has a PNH granulocyte clone greater than 50% of the total granulocyte count.

7. The method of claim 2, wherein the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

8. The method of claim 2, wherein the compound is selected from the group consisting of CR1, LEX-CRI, MCP, DAF, CD59, Factor H, cobra venom factor, FUT-175, complestatin, and K76 COOH.

9. The method of claim 2, wherein said compound inhibits C5b activity.

10. The method of claim 2, wherein said compound inhibits cleavage of C5.

11. The method of claim 2, wherein said compound inhibits terminal complement.

12. The method of claim 2, wherein said compound inhibits C5a activity or inhibits binding of C5a to its receptor.

13. The method of claim 1, wherein said subject is a human.

14. The method of claim 1, wherein said subject has a history of one or more thrombotic events.

15. The method of claim 7, wherein said compound is an antibody or antibody fragment.

16. The method of claim 15, wherein said antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

17. The method of claim 15, wherein said antibody is pexelizumab.

18. The method of claim 15, wherein said antibody is eculizumab.

19. The method of claim 2, wherein said compound is administered chronically to said subject.

20. The method of claim 2, wherein said compound is administered systemically to said subject.

21. The method of claim 2, wherein said compound is administered locally to said subject.

22. The method of claim 1, wherein said method reduces rates of thromboembolism by greater than 25%.

23. The method of claim 1, wherein said method reduces rates of thromboembolism by greater than 50%.

24. The method of claim 1, wherein said method reduces rates of thromboembolism by greater than 75%.

25. The method of claim 1, wherein said method reduces rates of thromboembolism by greater than 90%.

26. The method of claim 1, wherein said method results in at least a 25% reduction in LDH levels.

27. The method of claim 1, wherein said method results in at least a 50% reduction in LDH levels.

28. The method of claim 1, wherein said method results in at least a 75% reduction in LDH levels.

29. The method of claim 1, wherein said method in a subject results in at least a 90% reduction in LDH levels.

30. The method of claim 2, further comprising administering a second compound, wherein said second compound increases hematopoiesis.

31. The method of claim 30, wherein the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG).

32. The method of claim 31, wherein EPO is administered with an anti-C5 antibody.

33. The method of claim 32, wherein said antibody is pexelizumab.

34. The method of claim 32, wherein said antibody is eculizumab.

35. The method of claim 2, further comprising administering an antithrombotic compound.

36. The method of claim 35, wherein said antithrombotic compound is an anticoagulant.

37. The method of claim 36, wherein said anticoagulant is administered with an anti-C5 antibody.

38. The method of claim 36, wherein said anticoagulant is an antiplatelet agent.

39. The method of claim 37, wherein said antibody is pexelizumab.

40. The method of claim 37, wherein said antibody is eculizumab.

41. A method of reducing the occurrence of thrombosis in a subject who has a higher than normal lactate dehydrogenase (LDH) level, said method comprising inhibiting complement in said subject.

42. The method of claim 41 comprising administering a compound to said subject, wherein the compound is selected from the group consisting of: a) compounds which bind to one or more complement components, b) compounds which block the generation of one or more complement components, and c) compounds which block the activity of one or more complement components.

43. The method of claim 41, wherein said subject has an LDH level greater than the upper limit of normal.

44. The method of claim 41, wherein said subject has an LDH level greater than or equal to 1.5 times the upper limit of normal.

45. The method of claim 41, wherein said subject has an LDH level greater than or equal to 2.5 times the upper limit of normal.

46. The method of claim 41, wherein said subject has an LDH level greater than or equal to 5 times the upper limit of normal.

47. The method of claim 41, wherein said subject has an LDH level greater than or equal to 10 times the upper limit of normal.

48. The method of claim 42, wherein the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

49. The method of claim 42, wherein the compound is selected from the group consisting of CR1, LEX-CRl, MCP, DAF, CD59, Factor H, cobra venom factor, FUT-175, complestatin, and K76 COOH.

50. The method of claim 42, wherein said compound inhibits C5b activity.

51. The method of claim 42, wherein said compound inhibits cleavage of C5.

52. The method of claim 42, wherein said compound inhibits terminal complement.

53. The method of claim 42, wherein said compound inhibits C5a activity or inhibits binding of C5a to its receptor.

54. The method of claim 41, wherein said subject is a human.

55. The method of claim 41, wherein said subject has a history of one or more thrombotic events.

56. The method of claim 42, wherein said compound is an antibody or antibody fragment.

57. The method of claim 56, wherein said antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

58. The method of claim 56, wherein said antibody is pexelizumab.

59. The method of claim 56, wherein said antibody is eculizumab.

60. The method of claim 42, wherein said compound is administered chronically to said subject.

61. The method of claim 42, wherein said compound is administered systemically to said subject.

62. The method of claim 42, wherein said compound is administered locally to said subject.

63. The method of claim 41, wherein said method reduces rates of thromboembolism by greater than 25%.

64. The method of claim 41, wherein said method reduces rates of thromboembolism by greater than 50%.

65. The method of claim 41, wherein said method reduces rates of thromboembolism by greater than 75%.

66. The method of claim 41, wherein said method reduces rates of thromboembolism by greater than 90%.

67. The method of claim 41, wherein said method results in at least a 25% reduction in LDH levels.

68. The method of claim 41, wherein said method results in at least a 50% reduction in LDH levels.

69. The method of claim 41, wherein said method results in at least a 75% reduction in LDH levels.

70. The method of claim 41, wherein said method results in at least a 90% reduction in LDH levels.

71. The method of claim 42, further comprising administering a second compound, wherein said second compound increases hematopoiesis.

72. The method of claim 71, wherein the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG).

73. The method of claim 72, wherein EPO is administered with an anti-C5 antibody.

74. The method of claim 73, wherein said antibody is pexelizumab.

75. The method of claim 73, wherein said antibody is eculizumab.

76. The method of claim 42, further comprising administering an antithrombotic compound.

77. The method of claim 76, wherein said antithrombotic compound is an anticoagulant.

78. The method of claim 77, wherein said anticoagulant is administered with an anti-C5 antibody.

79. The method of claim 77, wherein said anticoagulant is an antiplatelet agent.

80. The method of claim 78, wherein said antibody is pexelizumab.

81. The method of claim 78, wherein said antibody is eculizumab.

82. A method of reducing the occurrence of thrombosis in a subject who has a PNH granulocyte clone and an LDH level greater than the upper limit of normal, said method comprising inhibiting complement in said subject.

83. The method of claim 82 comprising administering a compound to said subject, wherein the compound is selected from the group consisting of: a) compounds which bind to one or more complement components, b) compounds which block the generation of one or more complement components, and c) compounds which block the activity of one or more complement components.

84. The method of claim 82, wherein said subject has a PNH granulocyte clone greater than 0.1% of the total granulocyte count.

85. The method of claim 82, wherein said subject has a PNH granulocyte clone greater than 0.1% of the total granulocyte count.

86. The method of claim 82, wherein said subject has a PNH granulocyte clone greater than 1% of the total granulocyte count.

87. The method of claim 82, wherein said subject has a PNH granulocyte clone greater than 10% of the total granulocyte count.

88. The method of claim 82, wherein said subject has a PNH granulocyte clone greater than 50% of the total granulocyte count.

89. The method of claim 83, wherein the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

90. The method of claim 83, wherein the compound is selected from the group consisting of CR1, LEX-CRI, MCP, DAF, CD59, Factor H, cobra venom factor, FUT-175, complestatin, and K76 COOH.

91. The method of claim 83, wherein said compound inhibits C5b activity.

92. The method of claim 83, wherein said compound inhibits cleavage of C5.

93. The method of claim 83, wherein said compound inhibits terminal complement.

94. The method of claim 83, wherein said compound inhibits C5a activity or inhibits binding of C5a to its receptor.

95. The method of claim 82, wherein said subject is a human.

96. The method of claim 82, wherein said subject has a history of one or more thrombotic events.

97. The method of claim 89, wherein said compound is an antibody or antibody fragment.

98. The method of claim 97, wherein said antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

99. The method of claim 97, wherein said antibody is pexelizumab.

100. The method of claim 97, wherein said antibody is eculizumab.

101. The method of claim 83, wherein said compound is administered chronically to said subject.

102. The method of claim 83, wherein said compound is administered systemically to said subject.

103. The method of claim 83, wherein said compound is administered locally to said subject.

104. The method of claim 82, wherein said method reduces rates of thromboembolism by greater than 25%.

105. The method of claim 82, wherein said method reduces rates of thromboembolism by greater than 50%.

106. The method of claim 82, wherein said method reduces rates of thromboembolism by greater than 75%.

107. The method of claim 82, wherein said method reduces rates of thromboembolism by greater than 90%.

108. The method of claim 82, wherein said method results in at least a 25% reduction in LDH levels.

109. The method of claim 82, wherein said method results in at least a 50% reduction in LDH levels.

110. The method of claim 82, wherein said method results in at least a 75% reduction in LDH levels.

111. The method of claim 82, wherein said method results in at least a 90% reduction in LDH levels.

112. The method of claim 83, further comprising administering a second compound, wherein said second compound increases hematopoiesis.

113. The method of claim 112, wherein the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG).

114. The method of claim 113, wherein EPO is administered with an anti-C5 antibody.

115. The method of claim 114, wherein said antibody is pexelizumab.

116. The method of claim 114, wherein said antibody is eculizumab.

117. The method of claim 83, further comprising administering an antithrombotic compound.

118. The method of claim 117, wherein said antithrombotic compound is an anticoagulant.

119. The method of claim 118, wherein said anticoagulant is administered with an anti-C5 antibody.

120. The method of claim 118, wherein said anticoagulant is an antiplatelet agent.

121. The method of claim 119, wherein said antibody is pexelizumab.

122. The method of claim 119, wherein said antibody is eculizumab.

123. A method of reducing the occurrence of thrombosis in a subject suffering from a lower than normal nitric oxide (NO) level, said method comprising inhibiting complement in said subject.

124. The method of claim 123 comprising administering a compound to said subject, wherein the compound is selected from the group consisting of: i) compounds which bind to one or more complement components, ii) compounds which block the generation of one or more complement components, and iii) compounds which block the activity of one or more complement components, wherein said method increases serum nitric oxide (NO) levels.

125. The method of claim 123, wherein said method increases NO levels by greater than 25%.

126. The method of claim 123, wherein said method increases NO levels by greater than 50%.

127. The method of claim 123, wherein said method increases NO levels by greater than 100%.

128. The method of claim 123, wherein said method increases NO levels by greater than 3 fold.

129. The method of claim 123, wherein the subject has PNH.

130. The method of claim 124, wherein the compound is selected from the group consisting of antibodies, soluble complement inhibitory compounds, proteins, protein fragments, peptides, small molecules, RNA aptamers, L-RNA aptamers, spiegelmers, antisense compounds, serine protease inhibitors, double stranded RNA, small interfering RNA, locked nucleic acid inhibitors, and peptide nucleic acid inhibitors.

131. The method of claim 124, wherein the compound is selected from the group consisting of CR1, LEX-CRI, MCP, DAF, CD59, Factor H, cobra venom factor, FUT-175, complestatin, and K76 COOH.

132. The method of claim 124, wherein said compound inhibits C5b activity.

133. The method of claim 124, wherein said compound inhibits cleavage of C5.

134. The method of claim 124, wherein said compound inhibits terminal complement.

135. The method of claim 124, wherein said compound inhibits C5a activity or inhibits binding of C5a to its receptor.

136. The method of claim 123, wherein said subject is a human.

137. The method of claim 123, wherein said subject has a history of one or more thrombotic events.

138. The method of claim 130, wherein said compound is an antibody or antibody fragment.

139. The method of claim 138, wherein said antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, an Fd, and an F(ab′)₂.

140. The method of claim 138, wherein said antibody is pexelizumab.

141. The method of claim 138, wherein said antibody is eculizumab.

142. The method of claim 124, wherein said compound is administered chronically to said subject.

143. The method of claim 124, wherein said compound is administered systemically to said subject.

144. The method of claim 124, wherein said compound is administered locally to said subject.

145. The method of claim 123, wherein said method reduces rates of thromboembolism by greater than 25%.

146. The method of claim 123, wherein said method reduces rates of thromboembolism by greater than 50%.

147. The method of claim 123, wherein said method reduces rates of thromboembolism by greater than 75%.

148. The method of claim 123, wherein said method reduces rates of thromboembolism by greater than 90%.

149. The method of claim 123, wherein said method results in at least a 25% reduction in LDH levels.

150. The method of claim 123, wherein said method results in at least a 50% reduction in LDH levels.

151. The method of claim 123, wherein said method results in at least a 75% reduction in LDH levels.

152. The method of claim 123, wherein said method results in at least a 90% reduction in LDH levels.

153. The method of claim 124, further comprising administering a second compound, wherein said second compound increases hematopoiesis.

154. The method of claim 153, wherein the second compound is selected from the group consisting of steroids, immunosuppressants, anti-coagulants, folic acid, iron, erythropoietin (EPO), pegylated EPO, EPO mimetics, Aranesp®, erythropoiesis stimulating agents, antithymocyte globulin (ATG) and antilymphocyte globulin (ALG).

155. The method of claim 154, wherein EPO is administered with an anti-C5 antibody.

156. The method of claim 155, wherein said antibody is pexelizumab.

157. The method of claim 155, wherein said antibody is eculizumab.

158. The method of claim 124, further comprising administering an antithrombotic compound.

159. The method of claim 158, wherein said antithrombotic compound is an anticoagulant.

160. The method of claim 159, wherein said anticoagulant is administered with an anti-C5 antibody.

161. The method of claim 159, wherein said anticoagulant is an antiplatelet agent.

162. The method of claim 160, wherein said antibody is pexelizumab.

163. The method of claim 160, wherein said antibody is eculizumab.

164. A method of determining whether a subject having a hemolytic disorder is susceptible to thrombosis comprising measuring the PNH granulocyte clone size of said subject, wherein if the clone size is greater than 0.1% then said subject is susceptible to thrombosis.

165. The method of claim 164, wherein said clone size is greater than 1%.

166. The method of claim 164, wherein said clone size is greater than 10%.

167. The method of claim 164, wherein said clone size is greater than 50%.

168. A method of increasing PNH red blood cell mass of a subject, said method comprising inhibiting complement in said subject.

169. The method of claim 168 comprising administering a compound to the subject, the compound being selected from the group consisting of: i) compounds which bind to one or more complement components, ii) compounds which block the generation of one or more complement components, and iii) compounds which block the activity of one or more complement components.

170. The method of claim 168, wherein said subject has a PNH granulocyte clone.

171. The method of claim 170, wherein said PNH granulocyte clone is greater than 0.1% of the total granulocyte count.

172. The method of claim 170, wherein said PNH granulocyte clone is greater than I% of the total granulocyte count.

173. The method of claim 170, wherein said PNH granulocyte clone is greater than 10% of the total granulocyte count.

174. The method of claim 170, wherein said PNH granulocyte clone is greater than 50% of the total granulocyte count.

175. The method of claim 168, wherein said subject has an LDH level greater than the upper limit of normal.

176. The method of claim 175, wherein said subject has an LDH level greater than or equal to 1.5 times the upper limit of normal.

177. The method of claim 175, wherein said subject has an LDH level greater than or equal to 2.5 times the upper limit of normal.

178. The method of claim 175, wherein said subject has an LDH level greater than or equal to 5 times the upper limit of normal.

179. The method of claim 175, wherein said subject has an LDH level greater than or equal to 10 times the upper limit of normal.

180. A method of treating hemolytic anemia in a subject, said method comprising inhibiting complement in said subject.

181. The method of claim 180, wherein said method comprises administering a compound to the subject, wherein the compound is selected from the group consisting of: i) compounds which bind to one or more complement components, ii) compounds which block the generation of one or more complement components, and iii) compounds which block the activity of one or more complement components, wherein said method increases red blood cell (RBC) mass.

182. The method of claim 181, wherein RBC mass is measured as the absolute number of RBCs.

183. The method of claim 181, wherein RBC mass is PNH RBC mass.

184. The method of claim 183, wherein said method increases RBC mass by greater than 10%.

185. The method of claim 183, wherein said method increases RBC mass by greater than 25%.

186. The method of claim 183, wherein said method increases RBC mass by greater than 50%.

187. The method of claim 183, wherein said method increases RBC mass by greater than 100%.

188. The method of claim 183, wherein said method increases RBC mass by greater than 2 fold.

189. The method of claim 180, wherein said method decreases transfusion requirements.

190. The method of claim 180, wherein said method stabilizes hemoglobin levels.

191. The method of claim 180, wherein said method causes an increase in hemoglobin levels.
